(19) United States
(12) Patent Application Publication
(10) Pub. No.: US 2009/0069430 A1
(43) Pub. Date: Mar. 12, 2009

(54) DIBENZENE DERIVATIVES AS CALCIUM CHANNEL BLOCKERS

(75) Inventors: Ana MARTINEZ GIL, Madrid (ES); Ana Castro Morera, Madrid (ES); Miguel Medina Padilla, Madrid (ES); Pilar Munoz Ruiz, Madrid (ES); Laura Rubio Arrieta, Madrid (ES); Esther Garcia Palomero, Madrid (ES); Celia De Austria De Luque, Madrid (ES); Jorge Sanchez-Quesada, Madrid (ES); Daniel Ignacio Perez Fernandez, Madrid (ES); Javier Lopez Ogalla, Madrid (ES); Diana Alonso Gordillo, Madrid (ES)

Correspondence Address: MOORE & VAN ALLEN PLLC, P.O. BOX 13706, Research Triangle Park, NC, 27709, US

(73) Assignee: NEUROPHARMA, S.A., Madrid (ES)

(21) Appl. No.: 12/139,863

(22) Filed: Jun. 16, 2008

(30) Foreign Application Priority Data
Dec. 16, 2005 (EP) EP05077910.7
Jan. 3, 2006 (EP) EP06380004.9

(51) Int. Cl. A61K 31/166; C07C 233/01; A61P 25/16; A61P 25/18; A61P 25/32; A61P 25/28

(52) U.S. Cl. 514/616; 564/169

(57) ABSTRACT

The invention is directed to a compound of formula (I)

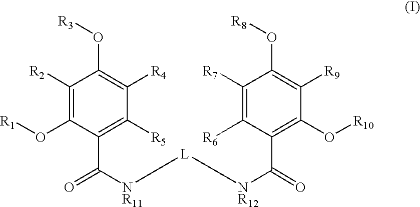

Having VDCC blocking activity. These compounds are useful for the treatment of a series of human diseases and conditions, especially cognitive or neurodegenerative diseases or conditions.

### FIELD OF THE INVENTION

This invention is related to a new family of synthetic compounds and to their use in the treatment of cognitive or neurodegenerative diseases, disorders or conditions.

### BACKGROUND OF THE INVENTION

Calcium Ca²⁺ concentrations and specially their fluctuation in different cellular subcompartments seem to be a universal signaling system, thus regulating most of the cellular functions, from contraction to gene expression through cell death.

The calcium ion is one of the most important elements in the physiological equilibrium of cells, acting not only as a neurotransmitter, but also as a second messenger. In order to control levels of calcium inside and outside cells, they are provided with different types of calcium channels. These channels control the calcium influx through the membrane where they are located, and they can be modulated by voltage changes or by ligands. The voltage-dependant Ca²⁺ channels (VDCCs) are an important type of calcium channels which are very numerous in cells with electrophysiological activity, such as neurons and muscle fibres cells. They consist of five subunits encoded by different groups of genes and designated as α₁ (the channel forming subunit), α₂δ, β, γ. The complex is provided with several sites for N-glycosylation and AMP-dependant protein kinases phosphorylation. When calcium enters the cytoplasma, it can bind different modulating proteins, to provoke diverse sequences of steps which in turn lead to different physiological changes. Calcium signaling pathways have several critical functions, such as nerve impulse transmission, muscle contraction, hormones secretion and constriction/relaxation of blood vessels.

However, an uncontrolled level of calcium can lead to different negative effects, such as neuronal excitotoxicity and other forms of cell death (Mechanisms of calcium-related cell death, Orrenius et al., Adv Neurol. 1996; 71:137-49). Excitotoxicity is an excessive release of neurotransmitters which damages cells of the CNS (Inciting excitotoxic cytocide among central neurons, Olney J. W., Adv Exp Med Biol. 1986; 203:631-45) and is often attributed to glutamate. An excessive synaptic release of glutamate can lead to the disregulation of Ca²⁺ homeostasis. Glutamate activates postsynaptic ionotropic receptors, such as NMDA or AMPA, which open their associated ionic channels to allow the influx of Ca²⁺ and other ions. Although the exact mechanism by which Ca²⁺ mediates excitotoxicity seems not to have been determined with complete certainty, some authors have hypothesized that it occurs following the activation of distinct signaling cascades downstream from key points of Ca²⁺ entry at synapses (Molecular mechanism of calcium-dependent neurodegeneration in excitotoxicity, Arundine M. and Tymianski M., Cell Calcium; 2003; 34 (4-5):325-327).

There are a lot of evidence that excitotoxicity may play a role in certain neuropathological events, such as neuronal death in stroke and ischemia, and in neurodegenerative diseases, such as Huntington's Disease (HD), Parkinson's Disease (PD) or Alzheimer's Disease (AD) (The role of excitotoxicity in neurodegenerative disease: implications for therapy, Doble A., Pharmacol Ther. 1999; 81(3):163-221; Glutamate-mediated excitotoxicity and neurodegeneration in Alzheimer's disease, Hynd M R, Scott H L, Dodd P R., Neurochem. Int. 2004; 45(5):583-95). Changes in the number and structure of VDCCs occur in the brain during the aging process, and these changes are closely associated with several development functions of cells. Thus, VDCCs could be involved in increasing the vulnerability of the CNS cells to excitotoxicity with age (Decreased G-Protein-Mediated Regulation and Shift in Calcium Channel Types with Age in Hippocampal Cultures, Landfield et al., J. Neurosci., 1999; 19(19):8674-8684).

Furthermore, the accumulation of amyloid-β-protein (Aβ) in the brain is a characteristic event in the pathology of Alzheimer's Disease. The processing of amyloid protein precursor (APP) results in the production of Aβ peptides with different numbers of aminoacids in their chains. These peptides have been found to be toxic to cells in culture because they disrupt calcium homeostasis in human cortical neurons (β-Amyloid peptides destabilize calcium homeostasis and render human cortical neurons vulnerable to excitotoxicity, Mattson M P et al., J Neurosci. 1992; 12(2):376-89), and this process may be mediated in part by the opening of certain VDCCs (Amyloid beta protein potentiates Ca2+ influx through L-type voltage-sensitive Ca2+ channels: a possible involvement of free radicals, Ueda K et al., J Neurochem. 1997; 68(1):265-71). Another physiological change in AD is an increase in acetylcholinesterase (AchE) activity around the amyloid accumulations, which leads to a loss of efficiency in both cholinergic and non-cholinergic neurons in the brain. The influx of calcium through certain VDCCs seems to have an effect on AChE expression because drugs acting as blockers of these Ca2+ channels, such as nifedipine, resulted in a decrease of AChE expression in cultured cells (The amyloid beta-protein of Alzheimer's disease increases acetylcholinesterase expression by increasing intracellular calcium in embryonal carcinoma P19 cells, Sberna G. et al., J Neurochem. 1997; 69(3): 1177-84).

Other publications have related disorders in the levels of Ca²⁺ in nerve cells with other diseases and disorders, especially cognitive and neurodegenerative diseases and disorders. This is the case, for example, of WO2005/097779, wherein the control of Ca²⁺ concentration in cells has been related to diseases such as stroke, anxiety (such as panic disorder, obsessive-compulsive disorder, post-traumatic stress syndrome), epilepsy, head trauma, migraine, chronic pain (such as cancer pain, inflammatory pain conditions related to osteoarthritis, rheumatoid arthritis and fibromyalgia), neuropathic pain (such as diabetic peripheral neuropathy, post-herpetic neuralgia, trigeminal neuralgia, cancer pain and AIDS related neuropathy) and acute pain (such as nocicceptive pain and post-operative pain, schizophrenia, depression, psychoses, drug and alcohol addiction, and neurodegenerative disorders (such as Parkinson's Disease, Alzheimer's Disease, multiple sclerosis, neuropathies, Huntington's Disease and amyotrophic lateral sclerosis (ALS)).

Therefore, taking into account that Ca²⁺ seems to have a direct implication in a series of important human diseases and disorders, especially cognitive and neurodegenerative disorders, there is a need for finding effective VDCCs blockers in order to control the levels of Ca²⁺ in nerve cells in order to obtain effective medicaments for the treatment of such diseases and disorders.

### SUMMARY OF THE INVENTION

A new family of compounds having VDCC blocking activity has been found. In a first aspect, the present invention is related to a compound of formula (I)

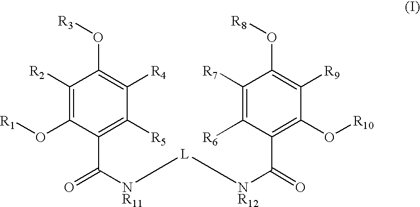

wherein
R₁ and R₁₀ are independently selected from hydrogen, substituted or unsubstituted alkyl, substituted or unsubstituted alkenyl, —(CH₂)ₘ—(CO)—Rₐ, —(CH₂)ₘ—(CO)—O—Rₐ or —(CH₂)ₘ—O—Ra, wherein m is an integer selected from 1 or 2 and Rₐ is selected from hydrogen, substituted or unsubstituted alkyl, substituted or unsubstituted cycloalkyl, substituted or unsubstituted alkenyl, substituted or unsubstituted aryl, substituted or unsubstituted aralkyl or substituted or unsubstituted heterocyclyl;
R₃ and R₈ are independently selected from hydrogen, substituted or unsubstituted alkyl, substituted or unsubstituted cycloalkyl, substituted or unsubstituted alkenyl, substituted or unsubstituted aryl, substituted or unsubstituted aralkyl, substituted or unsubstituted heterocyclyl, substituted or unsubstituted alkoxy, substituted or unsubstituted aryloxy or halogen;
R₁₁ and R₁₂ are independently selected from hydrogen, substituted or unsubstituted alkyl, substituted or unsubstituted cycloalkyl, substituted or unsubstituted alkenyl, substituted or unsubstituted aryl, substituted or unsubstituted aralkyl, substituted or unsubstituted heterocyclyl, substituted or unsubstituted alkoxy, substituted or unsubstituted aryloxy or halogen;
R₅ and R₆ are independently selected from hydrogen, C₁-C₆ alkoxy, C₁-C₆ alkyl or halogen, preferably Br;
R₂ and R₉ are independently selected from hydrogen, C₁-C₆ alkoxy, C₁-C₆ alkyl or halogen, preferably Br;
R₄ and R₇ are independently selected from hydrogen, C₁-C₆ alkoxy, C₁-C₆ alkyl or halogen, preferably Br;
L is a linker, consisting of a linear sequence of 1-20 units selected from —(CH₂)ₙ—, —CO—, —O—, —S—, substituted or unsubstituted arylene, cycloalkylene, heterocyclylene, or —NH—; n=1-10;
with the provisos that:
in L, two —NH— units may not be adjacent;
when L consists of a —(CH₂)ₙ— group then, n is 5-10;
or its enantiomers, diastereomers, tautomers, and pharmaceutically acceptable solvates and salts thereof.

Due to their VDCC blocking activity, these compounds may be useful for the treatment of a series of human diseases and conditions, especially cognitive or neurodegenerative diseases or conditions; therefore, according to another aspect, the present invention is related to the use of a compound of formula (I) as defined above in the preparation of a medicament for the treatment of a cognitive or neurodegenerative disease or condition.

In the frame of the present invention, the term “cognitive or neurodegenerative disease or condition” should be interpreted as including, but not being limited to, stroke, ischemia, anxiety, epilepsy, head trauma, migraine, chronic pain, neuropathic pain and acute pain, schizophrenia, depression, psychoses, drug and alcohol addiction, and neurodegenerative disorders such as Parkinson's Disease, Alzheimer's Disease, multiple sclerosis, neuropathies, Huntington's Disease and amyotrophic lateral sclerosis (ALS)).

A third aspect of the invention is a pharmaceutical composition which comprises at least one compound of formula (I) as defined above, or a pharmaceutically acceptable salt or solvate thereof, and a pharmaceutically acceptable carrier, adjuvant or vehicle.

The compounds of formula (I) according to the present invention may also be used as reactives for blocking VDCC in biological assays. Therefore, an additional aspect of the present invention is related to the use of the compounds of formula (I) as reactives for biological assays, preferably as reactives for blocking VDCC.

Another aspect of the present invention is a method of treating or preventing a disease or condition involving alterations of Ca²⁺ homeostasis, which method comprises administering to a patient in need of such a treatment a therapeutically effective amount of at least one compound of formula (I) as defined above or a pharmaceutical composition thereof.

### DETAILED DESCRIPTION OF THE INVENTION

In the above definition of compounds of formula (I) the following terms have the meaning indicated:

The term “alkenyl” refers to a linear, branched or cyclic hydrocarbon group of 2 to about 20 carbon atoms containing at least one double bond, such as ethenyl, n-propenyl, isopropenyl, n-butenyl, isobutenyl, octenyl, decenyl, tetradecenyl, hexadecenyl, eicosenyl, tetracosenyl, and the like. The term “substituted alkenyl” refers to alkenyl substituted with one or more substituent groups. The term “alkenyl” includes linear, branched, cyclic, unsubstituted, substituted, and/or heteroatom-containing alkenyl.

“Alkoxy” refers to a radical of the formula —ORa where Ra is an alkyl radical as defined below, e.g., methoxy, ethoxy, propoxy, etc.

“Alkyl” refers to a straight or branched hydrocarbon chain radical consisting of carbon and hydrogen atoms, containing no saturation, having one to eight carbon atoms, and which is attached to the rest of the molecule by a single bond, e.g., methyl, ethyl, n-propyl, i-propyl, n-butyl, t-butyl, n-pentyl, etc. Alkyl radicals may be optionally substituted. by one or more substituents.

“Aralkyl” refers to an alkyl group with an aryl substituent, wherein “alkyl” and “aryl” are as defined above. In general, aralkyl groups herein contain 6 to 24 carbon atoms, while preferred aralkyl and alkaryl groups contain 6 to 16 carbon atoms, and particularly preferred such groups contain 6 to 12 carbon atoms. Examples of aralkyl groups include, without limitation, benzyl, 2-phenyl-ethyl, 3-phenyl-propyl, 4-phenyl-butyl, 5-phenyl-pentyl, 4-phenylcyclohexyl, 4-benzylcyclohexyl, 4-phenylcyclohexylmethyl, 4-benzylcyclohexylmethyl, and the like. Alkaryl groups include, for example, p-methylphenyl, 2,4-dimethylphenyl, p-cyclohexylphenyl, 2,7-dimethylnaphthyl, 7-cyclooctylnaphthyl, 3-ethyl-cyclopenta-1,4-dienyl, and the like, preferably benzyl and phenethyl.

“Aryl” refers to an aromatic substituent generally containing 5 to 30 carbon atoms and containing a single aromatic ring or multiple aromatic rings that are fused together, directly linked, or indirectly linked (such that the different aromatic rings are bound to a common group such as a methylene or ethylene moiety). Preferred aryl groups contain 5 to 24 carbon atoms, and particularly preferred aryl groups contain 5 to 14 carbon atoms. Exemplary aryl groups contain one aromatic ring or two fused or linked aromatic rings, e.g., phenyl, naphthyl, biphenyl, diphenylether, diphenylamine, benzophenone, indenyl, fenanthryl or anthracyl and the like. “Substituted aryl” refers to an aryl moiety substituted with one or more substituent groups. If not otherwise indicated, the term “aryl” includes unsubstituted, substituted, and/or heteroatom-containing aromatic substituents.

The term “arylene” refers to a diradical derived from aryl or substituted aryl as defined above, and is exemplified by 1,2-phenylene, 1,3-phenylene, 1,4-phenylene, 1,2-naphthylene and the like.

The term “aryloxy” refers to the group aryl —O— wherein the aryl group is as defined above including optionally substituted aryl groups as also defined above.

“Cycloalkyl” refers to a stable 3- to 10-membered monocyclic or bicyclic radical which is saturated or partially saturated, and which consist solely of carbon and hydrogen atoms. Unless otherwise stated specifically in the specification, the term “cycloalkyl” is meant to include cycloalkyl radicals which are optionally substituted by one or more substituents.

“Cycloalkylene” refers to a diradical derived from cycloalkyl as defined above, being optionally substituted.

The terms “halo,” “halide,” and “halogen” refer to a chloro, bromo, fluoro, or iodo substituent.

The terms “heterocycle”, “heterocyclyl”, or “heterocyclic” refers to a stable 3- to 15 membered ring which consists of carbon atoms and from one to five heteroatoms selected from the group consisting of nitrogen, oxygen, and sulfur, preferably a 4- to 8-membered ring with one or more heteroatoms, more preferably a 5- or 6-membered ring with one or more heteroatoms. For the purposes of this invention, the heterocycle may be a monocyclic, bicyclic or tricyclic ring system, which may include fused ring systems; and the nitrogen, carbon or sulfur atoms in the heterocyclyl radical may be optionally oxidised; the nitrogen atom may be optionally quaternized; and the heterocyclyl radical may be partially or fully saturated or aromatic. Examples of such heterocycles include, but are not limited to, azepines, benzimidazole, benzothiazole, furan, isothiazole, imidazole, indole, piperidine, piperazine, purine, quinoline, thiadiazole, tetrahydrofuran.

“Heterocyclylene” refers to a diradical derived from heterocylyl as defined above; it may optionally be substituted by one or more substituents.

References herein to substituted groups in the compounds of the present invention refer to the specified moiety that may be substituted at one or more available positions by one or more suitable groups, e.g., halogen such as fluoro, chloro, bromo and iodo; cyano; hydroxyl; nitro; azido; alkanoyl such as a C1-C6 alkanoyl group such as acyl and the like; carboxamido; alkyl groups including those groups having 1 to about 12 carbon atoms or from 1 to about 6 carbon atoms and more preferably 1-3 carbon atoms; alkenyl and alkynyl groups including groups having one or more unsaturated linkages and from 2 to about 12 carbon or from 2 to about 6 carbon atoms; alkoxy groups having one or more oxygen linkages and from 1 to about 12 carbon atoms or 1 to about 6 carbon atoms; aryloxy such as phenoxy; alkylthio groups including those moieties having one or more thioether linkages and from 1 to about 12 carbon atoms or from 1 to about 6 carbon atoms; alkylsulfinyl groups including those moieties having one or more sulfinyl linkages and from 1 to about 12 carbon atoms or from 1 to about 6 carbon atoms; alkylsulfonyl groups including those moieties having one or more sulfonyl linkages and from 1 to about 12 carbon atoms or from 1 to about 6 carbon atoms; aminoalkyl groups such as groups having one or more N atoms and from 1 to about 12 carbon atoms or from 1 to about 6 carbon atoms; carbocylic aryl having 6 or more carbons, particularly phenyl or naphthyl and aralkyl such as benzyl. Unless otherwise indicated, an optionally substituted group may have a substituent at each substitutable position of the group, and each substitution is independent of the other.

In the above-detailed formula (I), R₃ and R₈ are preferably a C₁-C₆ alkyl, being the same or different. Even more preferably, R₃ and R₈ are both methyl.

According to a preferred embodiment, the linker L consists of a —(CH₂)₅₋₁₀ group.

According to another preferred embodiment, the linker L comprises an ether unit (—O—) subsequent to a substituted or unsubstituted arylene unit. Preferably, the arylene unit is a substituted or unsubstituted benzylene unit.

A preferred group of compounds are those wherein the linker L has the formula (II)

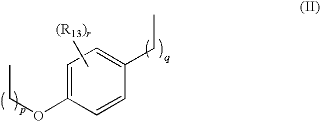

wherein R₁₃ is hydrogen or halogen, r is an integer selected from 1, 2 and 3; and p and q are integers independently selected from 1, 2, 3, 4 and 5.

Also preferred are compounds of formula (I) wherein R₅ and R₆ are both hydrogen.

According to another preferred embodiment, R₁₁ and R₁₂ are both hydrogen.

Another group of preferred compounds are those wherein at least one of R₂, R₄, R₇ and R₉ is a halogen, preferably Br.

A further group of preferred compounds are those wherein R₁ is equal to R₁₀, R₂ is equal to R₉, R₃ is equal to R₈, R₄ is equal to R₇, R₅ is equal to R₆.

Preferably, also the linker L is symmetric, the compounds having a symmetry plane.

In a preferred embodiment, R₄ is C₁-C₆ alkoxy.

The following are preferred compounds of formula (I) according to the present invention:

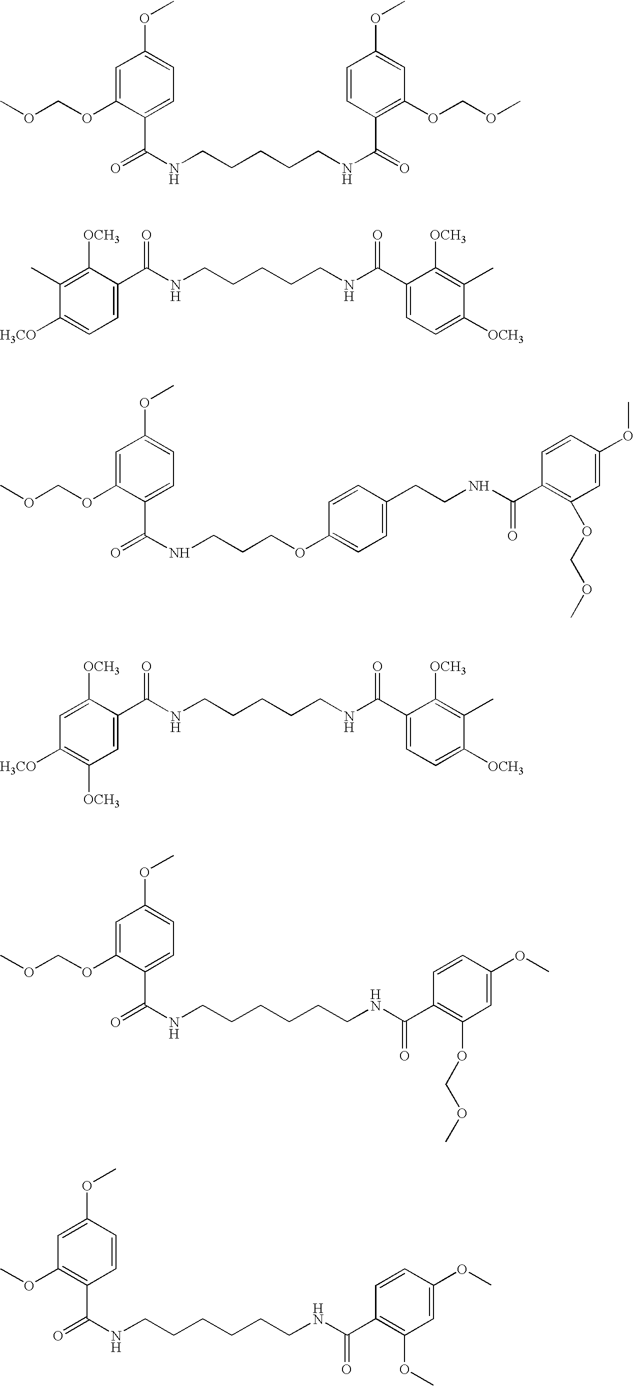

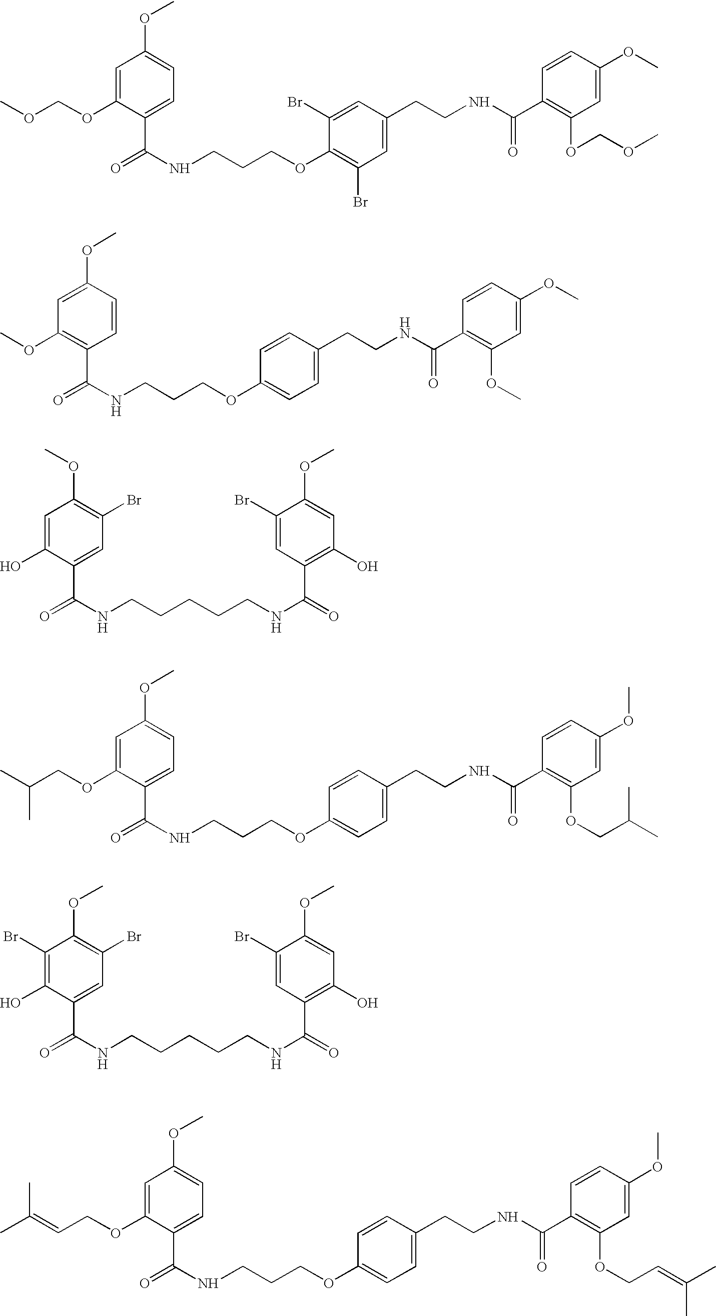

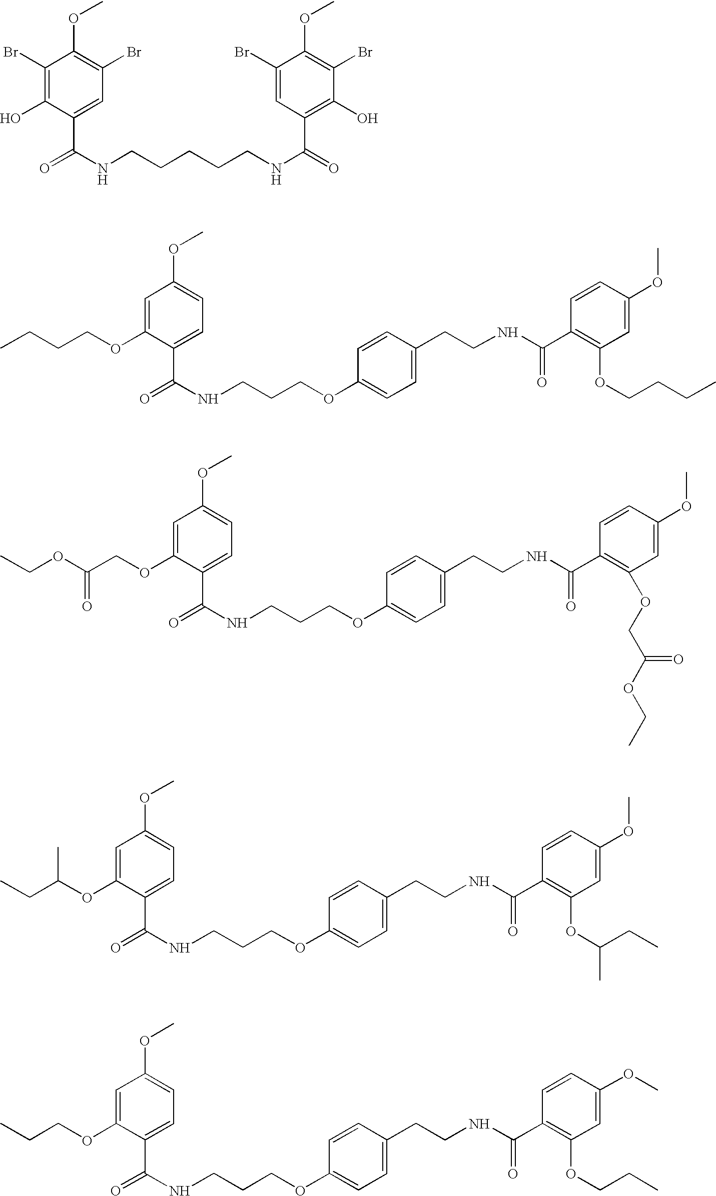

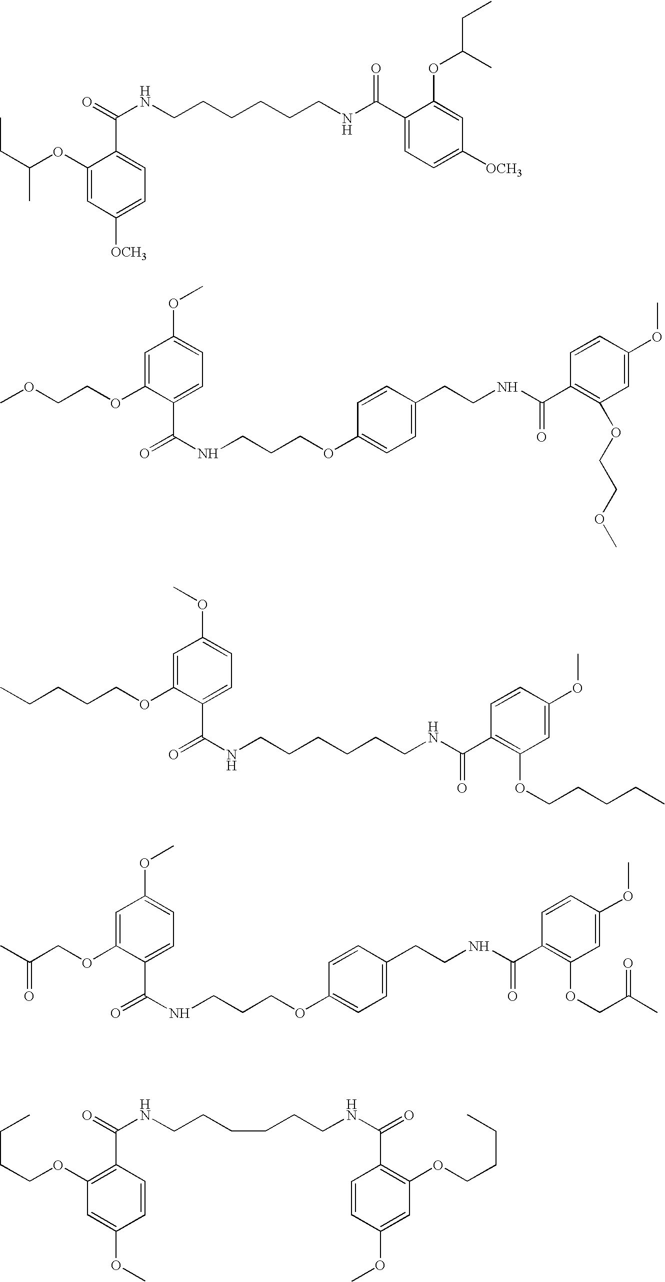

Unless otherwise stated, the compounds of the invention are also meant to include compounds which differ only in the presence of one or more isotopically enriched atoms. For example, compounds having the present structures except for the replacement of a hydrogen by a deuterium or tritium, or the replacement of a carbon by a 13C- or 14C-enriched carbon or 15N-enriched nitrogen are within the scope of this invention.

The term “pharmaceutically acceptable salts or solvates” refers to any pharmaceutically acceptable salt, ester, solvate, or any other compound which, upon administration to the recipient is capable of providing (directly or indirectly) a compound as described herein. However, it will be appreciated that non-pharmaceutically acceptable salts also fall within the scope of the invention since those may be useful in the preparation of pharmaceutically acceptable salts. The preparation of salts and derivatives can be carried out by methods known in the art.

For instance, pharmaceutically acceptable salts of compounds provided herein are synthesized from the parent compound which contains a basic or acidic moiety by conventional chemical methods. Generally, such salts are, for example, prepared by reacting the free acid or base forms of these compounds with a stoichiometric amount of the appropriate base or acid in water or in an organic solvent or in a mixture of the two. Generally, nonaqueous media like ether, ethyl acetate, ethanol, isopropanol or acetonitrile are preferred. Examples of the acid addition salts include mineral acid addition salts such as, for example, hydrochloride, hydrobromide, hydroiodide, sulphate, nitrate, phosphate, and organic acid addition salts such as, for example, acetate, maleate, fumarate, citrate, oxalate, succinate, tartrate, malate, mandelate, methanesulphonate and p-toluenesulphonate. Examples of the alkali addition salts include inorganic salts such as, for example, sodium, potassium, calcium, ammonium, magnesium, aluminium and lithium salts, and organic alkali salts such as, for example, ethylenediamine, ethanolamine, N,N-dialkylenethanolamine, triethanolamine, glucamine and basic aminoacids salts.

Particularly favoured derivatives are those that increase the bioavailability of the compounds of this invention when such compounds are administered to a patient (e.g., by allowing an orally administered compound to be more readily absorbed into the blood) or which enhance delivery of the parent compound to a biological compartment (e.g., the brain or lymphatic system) relative to the parent species.

The compounds of the invention may be in crystalline form either as free compounds or as solvates (e.g. hydrates) and it is intended that both forms are within the scope of the present invention. Methods of salvation are generally known within the art. Suitable solvates are pharmaceutically acceptable solvates. In a particular embodiment the solvate is a hydrate.

The compounds of formula (I) or their salts or solvates are preferably in pharmaceutically acceptable or substantially pure form. By pharmaceutically acceptable form is meant, inter alia, having a pharmaceutically acceptable level of purity excluding normal pharmaceutical additives such as diluents and carriers, and including no material considered toxic at normal dosage levels. Purity levels for the drug substance are preferably above 50%, more preferably above 70%, most preferably above 90%. In a preferred embodiment it is above 95% of the compound of formula (I), or of its salts, solvates or prodrugs.

The compounds of the present invention represented by the above described formula (I) may include enantiomers depending on the presence of chiral centres or isomers depending on the presence of multiple bonds (e.g. Z, E). The single isomers, enantiomers or diastereoisomers and mixtures thereof fall within the scope of the present invention.

In another aspect, the present invention is referred to a compound of formula (I) as defined above, for use as a medicament.

A further aspect of the invention is the use of a compound of formula (I) as defined above in the preparation of a medicament for the treatment of a cognitive or neurodegenerative disease, disorder or condition.

In the frame of the present invention, the term “cognitive or neurodegenerative disease or condition” should be interpreted as including, but not being limited to, stroke, ischemia, anxiety, epilepsy, head trauma, migraine, chronic pain, neuropathic pain and acute pain, schizophrenia, depression, psychoses, drug and alcohol addiction, and neurodegenerative disorders such as Parkinson's Disease, Alzheimer's Disease, multiple sclerosis, neuropathies, Huntington's Disease and amyotrophic lateral sclerosis (ALS)). Anxiety includes but is not limited to panic disorder, obsessive-compulsive disorder and post-traumatic stress syndrome; chronic pain includes but is not limited to cancer pain, inflammatory pain conditions related to osteoarthritis, rheumatoid arthritis and fibromyalgia; neuropathic pain includes but is not limited to diabetic peripheral neuropathy, post-herpetic neuralgia, trigeminal neuralgia, cancer pain and AIDS related neuropathy; acute pain includes but is not limited to nociceptive pain and post-operative pain.

More preferably, the cognitive or neurodegenerative disease, disorder or condition is Alzheimer's Disease. In another embodiment the disease or condition is epilepsy.

According to another aspect of the present invention, it is referred to a pharmaceutical composition which comprises at least one compound of formula (I) as defined above or a pharmaceutically acceptable salt or solvate thereof, and a pharmaceutically acceptable carrier, adjuvant or vehicle.

Examples of pharmaceutical compositions include any solid (tablets, pills, capsules, granules etc.) or liquid (solutions, suspensions or emulsions) composition for oral, topical or parenteral administration.

In a preferred embodiment the pharmaceutical compositions are in oral form. Suitable dose forms for oral administration may be tablets and capsules and may contain conventional excipients known in the art such as binding agents, for example syrup, acacia, gelatin, sorbitol, tragacanth, or polyvinylpyrrolidone; fillers, for example lactose, sugar, maize starch, calcium phosphate, sorbitol or glycine; tabletting lubricants, for example magnesium stearate; disintegrants, for example starch, polyvinylpyrrolidone, sodium starch glycollate or microcrystalline cellulose; or pharmaceutically acceptable wetting agents such as sodium lauryl sulfate.

The solid oral compositions may be prepared by conventional methods of blending, filling or tabletting. Repeated blending operations may be used to distribute the active agent throughout those compositions employing large quantities of fillers. Such operations are conventional in the art. The tablets may for example be prepared by wet or dry granulation and optionally coated according to methods well known in normal pharmaceutical practice, in particular with an enteric coating.

The pharmaceutical compositions may also be adapted for parenteral administration, such as sterile solutions, suspensions or lyophilized products in the appropriate unit dosage form. Adequate excipients can be used, such as bulking agents, buffering agents or surfactants.

The mentioned formulations will be prepared using standard methods such as those described or referred to in the Spanish and US Pharmacopoeias and similar reference texts.

Administration of the compounds or compositions of the present invention may be by any suitable method, such as intravenous infusion, oral preparations, and intraperitoneal and intravenous administration. Oral administration is preferred because of the convenience for the patient and the chronic character of many of the diseases to be treated.

Generally an effective administered amount of a compound of the invention will depend on the relative efficacy of the compound chosen, the severity of the disorder being treated and the weight of the sufferer. However, active compounds will typically be administered once or more times a day for example 1, 2, 3 or 4 times daily, with typical total daily doses in the range of from 0.1 to 1000 mg/kg/day.

The compounds and compositions of this invention may be used with other drugs to provide a combination therapy. The other drugs may form part of the same composition, or be provided as a separate composition for administration at the same time or at different time.

Taking into account that the compounds of formula (I) exhibit an inhibitory effect on VDCCs, the compounds may be used as reactives for biological assays, especially as reactives for blocking VDCCs. Therefore, another aspect of the invention is the use of a compound of formula (I) as defined above, or any salt or solvate thereof, as reactives for biological assays, preferably as a reactive for blocking VDCC.

A further aspect of the invention is a method of treating or preventing a disease, disorder or condition involving alterations of Ca²⁺ homeostasis, which method comprises administering to a patient in need of such a treatment a therapeutically effective amount of a compound of formula (I) as defined above, or any salt or solvate thereof, or a pharmaceutical composition thereof.

Final compounds of formula (I) according to the present invention can be obtained by a convergent pathway strategy which consist on coupling the conveniently substituted benzoic acid intermediate to the corresponding alkylic or arylic amines employing the methodology previously described by Padwa, A. et al, Synthesis, 1994, 9, 993-1004. The benzoic acid intermediate was obtained following synthetic standard procedures widely reported in the literature. While alkylic diamines are commercially available from Sigma-Aldrich the arylic amines were obtained from tyramine following similar reported literature procedures (Schoenfeld, R. C.; Conova, S.; Rittschof, D. and Ganem, B. Bioorganic & Medicinal Chemistry Letters 2002, 12, 823-825).

The following examples are given as further illustration of the invention, they should in no case be taken as a definition of the limits of the invention.

### EXAMPLES

### Preparation of the Compounds

Compounds of formula (I) according to the present invention were prepared following the general preparation strategy detailed above. Concretely, 22 compounds, named within this invention Compounds 1 to 22, with structures as detailed in Table 1, were synthesized.

TABLE 1

| Compound No. | Structure |
|---|---|
| 1 | 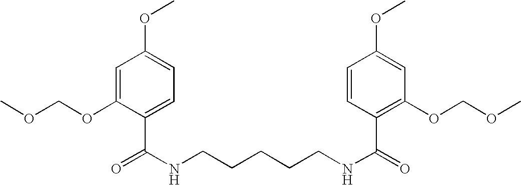 |
| 2 | 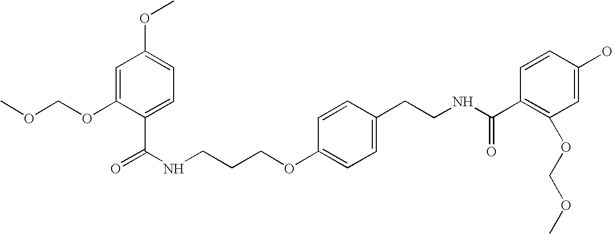 |
| 3 | 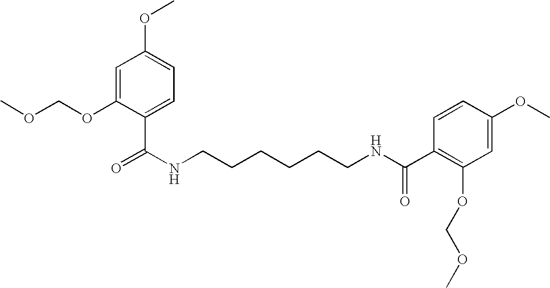 |
| 4 | 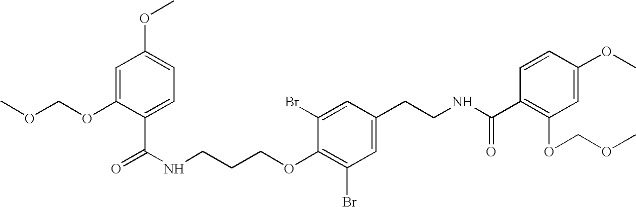 |
| 5 | 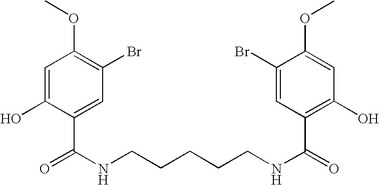 |
| 6 | 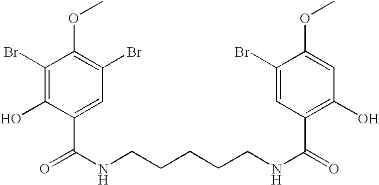 |
| 7 | 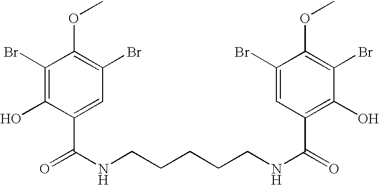 |
| 8 | 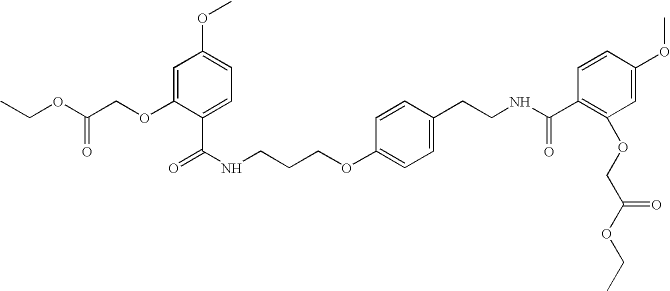 |
| 9 | 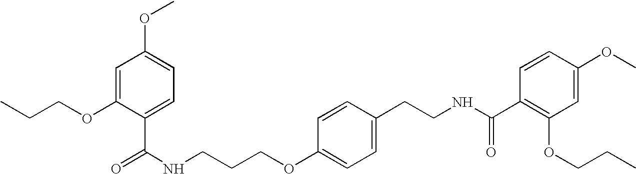 |
| 10 | 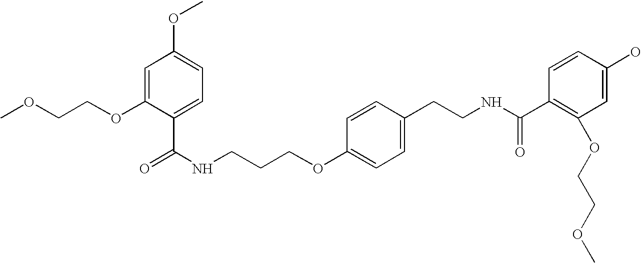 |
| 11 | 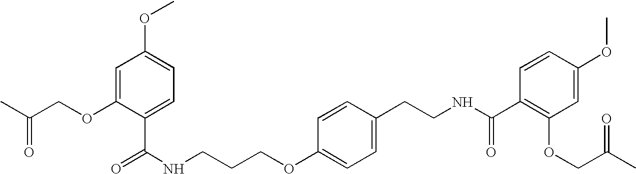 |
| 12 | 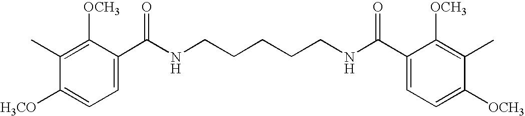 |
| 13 | 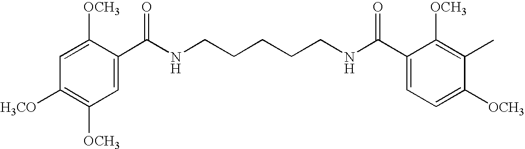 |
| 14 | 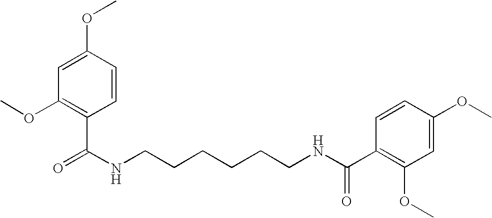 |
| 15 | 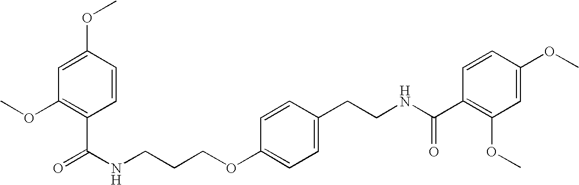 |
| 16 | 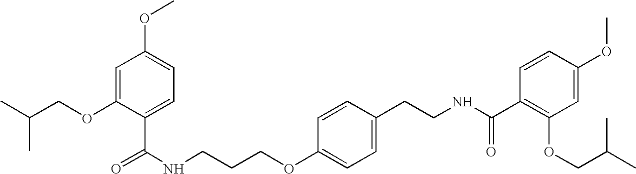 |
| 17 | 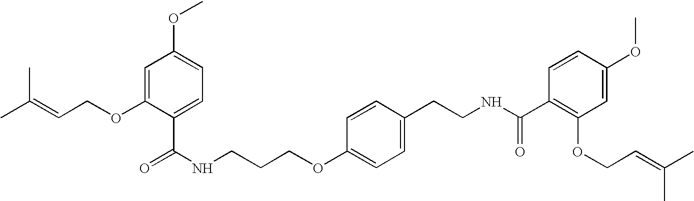 |
| 18 | 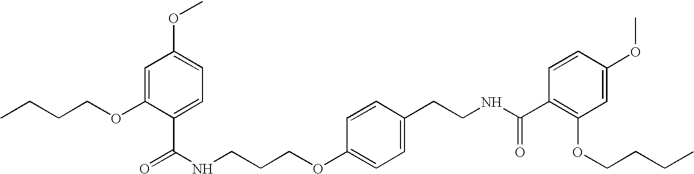 |
| 19 | 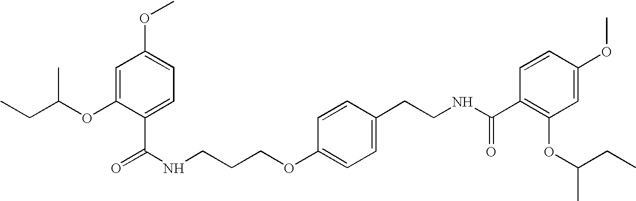 |
| 20 | 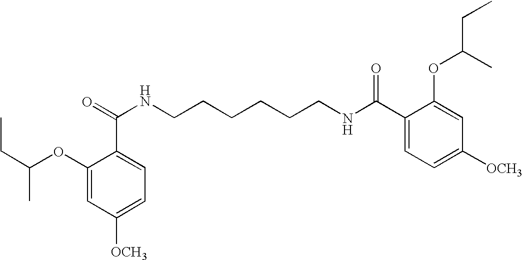 |
| 21 | 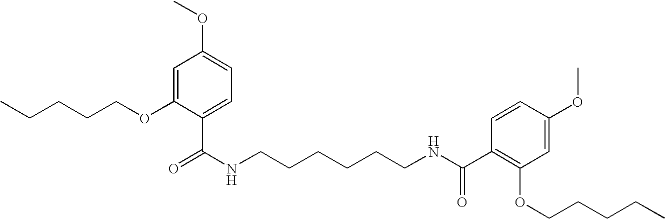 |
| 22 | 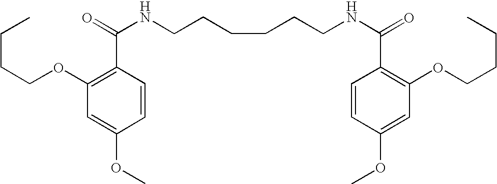 |

### Examples 1-4

### Preparation of Compounds 1, 2, 3, 4

The Compounds 1-4 were prepared according to the following general method:

To a solution of 2-(methoxymethoxy)-4-(methoxy)benzoic acid in anhydrous THF, 1,1′-carbonyldiimidazol was added under N₂ atmosphere, and the resulting mixture was stirred for four hours at room temperature. Afterwards, a solution of the corresponding diamines in anhydrous THF (DMF was also added when the corresponding diamine was not soluble in THF), and TEA (2 eq, only when the diamine was used as its trifluoroacetic salt) was added and the reaction mixture was stirred for further 20 hours. After evaporation of the solvent under reduced pressure, water was added and the resulting mixture was extracted with DCM. The combined organic extracts were washed with saturated NaCl solution and dried with Na₂SO₄. Evaporation of the solvent under reduced pressure gave a residue which was purified by silica gel flash-column chromatography as indicated below for each case, giving Compounds 1-4.

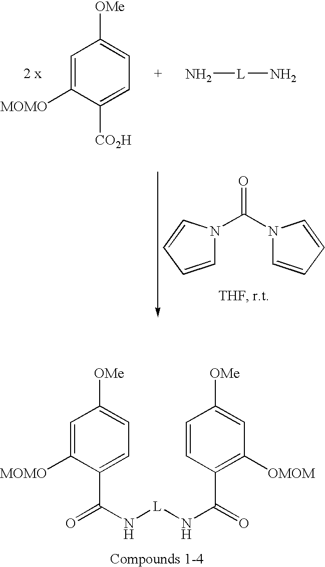

The intermediates which are necessary for this general procedure may be prepared as follows:

### Synthesis of the intermediate 2-(methoxymethoxy)-4-(methoxy)benzoic acid

A mixture of 2-hydroxy-4-methoxy-benzoic acid (5.0 g, 29.3 mmol) in MeOH (150 mL) and H₂SO₄ (2 mL) was refluxed for 48 hours. After evaporation of the solvent to reduced pressure, DCM (100 mL) was added and the solution was washed with water (100 mL), 10% K₂CO₃ solution, saturated NaCl solution and subsequently dried (Na₂SO₄), to give 4.8 g of the 2-hydroxy-4-methoxy-benzoic acid methyl ester derivative (89%) as a white solid.

A solution of this compound (4.8 g, 26.2 mmol) in anhydrous THF (24 mL) at 0° C. was treated with DIPEA (5.76 mL, 32.9 mmol) and subsequently with methoxymethyl chloride (2.47 mL, 32.9 mmol) over a period of 10 minutes. The reaction mixture was left to further stir at room temperature for 24 hours. Diethyl ether (200 mL) was added and the resulting solution was washed with water (2×100 mL), 0.1 M HCl solution (2×100 mL) and subsequently dried (Na₂SO₄), to give a residue which was purified by column chromatography (eluent used; hexane:ethyl acetate from 10:1 to 5:1), to give 5.9 g (88%) 2-(methoxymethoxy)-4-(methoxy)benzoic acid methyl ester.

The latter (4.4 g, 19.6 mmol) was hydrolysed by treatment with lithium hydroxide monohydrate (4.1 g, 97.9 mmol) in water/THF 1:3 (150 mL) for 3 days. THF was evaporated and the water phase cooled in an ice-bath, was neutralised to pH 3-4 with 0.1 M HCl solution, and extracted with DCM (4×50 mL). The combined extracts were dried (Na₂SO₄) and the solvent evaporated, to give 3.5 g (85%) of the 2-(methoxymethoxy)-4-(methoxy)benzoic acid as a white solid.

### Synthesis of the Arylic Diamine Intermediates

### Synthesis of 3-[4-(2-aminoethyl)-phenoxy]-propylamine diacetate salt

To a solution of tyramine (4-(2-aminoethyl)-phenol) (2.0 g, 14.6 mmol) in anhydrous DCM (30 mL), TEA (4.06 mL, 29.2 mmol) was added at room temperature. BOC anhydride (1.9 g, 8.76 mmol) was slowly added at 0° C. and the resulting mixture was stirred at room temperature for 2 days. DCM (50 mL) was added and the organic phase was washed with 0.1 M HCl (50 mL), water (3×100 mL), saturated NaCl solution, and subsequently dried (Na₂SO₄), and the solvent evaporated under reduced pressure, providing 2.15 g (61%) of [2-(4-hydroxy-phenyl)-ethyl]-carbamic acid tert-butyl ester.

A mixture of the above tert-butyl ester derivative (15.6 g, 66.0 mmol), N-(3-bromopropyl)-phtalimide (12.7 g, 66.0 mmol), K₂CO₃ (22.8 g, 132 mmol) and KI (3.29 g, 19.8 mmol) in acetonitrile was refluxed for 24 hours. The solvent was evaporated to dryness, water was added (300 mL), and the resulting mixture was extracted with DCM (2×300 mL). The combined extracts were washed with saturated NaCl solution, dried (Na₂SO₄) and the solvent removed. The resulting product was triturated in acetonitrile and filtered to give 20.2 g (72%) of (2-{4-[3-(1,3-dioxo-1,3-dihydroisoindole-2-yl)propoxy]-phenyl-ethyl)-carbamic acid tert-butyl ester.

A mixture of the above compound (20.2 g, 48 mmol) with hydrazine monohydrate (6.8 mL, 140 mmol) in MeOH (400) was refluxed for 4 hours. After evaporation of the solvent, the white solid obtained was suspended in DCM and the mixture cooled in an ice-bath. Filtration of the white precipitate gave 11.53 g (86%) of 3-[4-(2-amino-propoxy)-phenylamino]-propionic acid tert-butyl ester.

Treatment of this compound (1.9 g, 7.0 mmol) with TFA (25 mL) in THF (75 mL) at room temperature for 24 hours gave 2.80 g (93%) of 3-[4-(2-aminoethyl)-phenoxy]-propylamine as diacetate salt.

### Synthesis of 3-[4-(2-aminoethyl)-2,6-dibromo-phenoxy]-propylamine diacetate salt

It was synthesised from 4-(2,6-dibromo-2-aminoethyl)-phenol employing the same methodology described above. 4-(2,6-Dibromo-2-aminoethyl)-phenol was obtained by bromination of 4-(2-aminoethyl)-phenol. Thus, a solution of 4-(2-aminoethyl)-phenol (2 g, 14.6 mmol) in CHCl₃ (80 mL) was treated with pyridinium tribromide (9.3 g, 29.19 mmol) in pyridine (21 mL) for 24 hours and the solvent was evaporated to dryness. The brown solid obtained was suspended in water and cooled into an ice-bath. The precipitated was filtered and dried to give 4.14 g (98%) of 4-(2,6-dibromo-2-aminoethyl)-phenol. Structural characterization data are in accordance with that in the literature (Scheuer, P. J. and Hamann, M. T., J. Org. Chem. 1993, 58, 6565-6569).

### Example 1

### Preparation of Compound 1

Reagents: 2-(methoxymethoxy)-4-(methoxy)-benzoic acid (150 mg, 0.71 mmol) in anhydrous THF (5 mL); 1,1′-carbonyldiimidazol (120 mg, 0.74 mmol); 1,5-diaminopentane (50 μl, 0.42 mmol) in THF (5 mL).

Purification: silica gel flash-chromatography using EtOAc:MeOH (20:1).

Yield: 28 mg (16%) as a white solid.

¹H-NMR (CDCl₃, 400 MHz, [symbol] ppm): (8.10, d, 2H, J=8.6 Hz), (7.71, brs, 2H, NH), (6.60, d, 2H, J=2.3 Hz), (6.64, dd, 2H, J=2.3 Hz, J=8.6 Hz), (5.24, s, 4H), (3.79, s, 6H), (3.46, s, 6H), (3.43, m, 4H), (1.64, m, 4H), (1.47, m, 2H).

¹³C-NMR (CDCl₃, 100 MHz, [symbol] ppm): 165.1, 163.1, 156.5, 133.7, 115.3, 107.0, 101.3, 95.2, 56.8, 55.6, 39.6, 29.5, 24.6.

ESI-MS[M+H]⁺ 491.01.

### Example 2

### Preparation of Compound 2

Reagents: 2-(methoxymethoxy)-4-(methoxy)-benzoic acid (3.4 g, 15.8 mmol), anhydrous THF (20 mL); 1,1′-carbonyldiimidazol (2.7 g, 16.6 mmol); 3-[4-(2-aminoethyl)-phenoxy]-propylamine diacetate salt (4.0 g, 9.5 mmol) and TEA (4.6 mL, 5.17 mmol) in THF/DMF (12 mL, 1:1).

Purification: was not required.

Yield: 4.6 g (84%) as white solid.

¹H-NMR (CDCl₃, 400 MHz, δ ppm): (8.14, t, 2H, J=5.4 Hz), (8.00, t, 2H, J=5.4 Hz), (7.79, d, 1H, J=8.8 Hz), (7.73, d, 1H, J=8.8 Hz), (7.17, d, 2H, J=8.0 Hz), (6.88, d, 2H, J=8.0 Hz), (6.69, t, 2H, J=5.2 Hz), (6.65, s, 2H), (5.29, s, 2H), (5.25, s, 2H), (4.01, d, 2H, J=5.6 Hz), (3.77, s, 6H), (3.50-3.46, m, 4H), (3.35, s, 3H), (3.30, s, 3H), (2.76, t, 2H, J=6.8 Hz), (1.94, m, 2H).

¹³C-NMR (CDCl₃, 100 MHz, [symbol] ppm): 164.56, 164.2, 162.2, 162.1, 157.08, 155.9, 155.8, 132.2, 131.9, 131.3, 129.5, 116.6, 115.9, 114.4, 106.9, 106.8, 101.3, 101.2, 94.6, 94.4, 65.5, 56.0, 56.0, 55.4, 40.6, 36.2, 34.1, 30.6, 28.9. ESI-MS[M]⁺ 582.9.

### Example 3

### Preparation of Compound 3

Reagents: 2-(methoxymethoxy)-4-(methoxy)-benzoic acid (500 mg, 2.3 mmol) in anhydrous THF (10 mL); 1,1′-carbonyldiimidazol (400 mg, 2.5 mmol); 1,6-diaminohexane (164 mg, 1.41 mmol) in THF (4 mL).

Purification: silica gel flash-chromatography using EtOAc:MeOH (100:1).

Yield: 426 mg (72%) as a white solid.

¹H-NMR (CDCl₃, 400 MHz, [symbol] ppm): (8.12, d, 2H, J=9.0 Hz), (7.69, brs, 2H, NH), (6.65, d, 2H, J=2.3 Hz), (6.61, dd, 2H, J=2.3 Hz, J=9.0 Hz), (5.27, s, 4H), (3.80, s, 6H), (3.49, s, 6H), (3.42, m, 4H), (1.68-1.58, m, 4H), (1.44-1.41, m, 4H).

¹³C-NMR (CDCl₃, 100 MHz, [symbol] ppm): 165.1, 163.1, 156.5, 133.7, 115.6, 107.2, 101.5, 95.4, 56.8, 55.6, 39.7, 29.8, 26.9.

ESI-MS[M]⁺ 505.05.

### Example 4

### Preparation of Compound 4

Reagents: 2-(methoxymethoxy)-4-(methoxy)-benzoic acid (500 mg, 2.3 mmol), anhydrous THF (10 mL); 1,1′-carbonyldiimidazol (400 mg, 2.5 mmol); 3-[4-(2-aminoethyl)-2,6-dibromo-phenoxy]-propylamine diacetate salt (817 mg, 1.4 mmol) and TEA (0.7 mL, 5.17 mmol) in THF/DMF (11 mL, 10:1).

Purification: EtOAc:hexane (4:1).

Yield: 547 mg (63%) as white solid.

1H-NMR (CDCl₃, 400 MHz, [symbol] ppm): (8.15, d, 1H, J=6.3 Hz), (8.14, brs, 1H), (8.13, d, 1H, J=6.3 Hz), (7.73, brs, 1H), (7.40, s, 2H), (6.69, d, 1H, J=2.4 Hz), (6.66, d, 1H, J=2.4 Hz), (6.64-6.60, m, 2H), (5.17, s, 2H), (5.16, s, 2H), (4.12-4.07, m, 2H), (3.81, s, 6H), (3.78, q, 2H, J=6.2 Hz), (3.67, q, 2H, J=5.8 Hz), (3.38, s, 3H), (3.35, s, 3H), (2.84, t, 2H, J=6.8 Hz), (2.15, m, 2H).

¹³C-NMR (CDCl₃, 100 MHz, [symbol] ppm): 171.1, 165.1, 165.0, 163.3, 163.0, 156.7, 156.5, 151.5, 138.5, 133.6, 133.0, 118.2, 115.3, 114.8, 107.1, 107.0, 101.4, 95.2, 95.0, 60.3, 56.5, 56.3, 55.5, 55.5, 40.5, 37.5, 34.6, 29.9, 14.2.

ESI-MS[M]⁺ 740.9.

The Compounds 5-7 were prepared starting from Compound 1, according to the following procedure:

Compound 1 (176 mg, 0.4 mmol) in DCM (5 mL) was dropwise added to a solution of pyridinium tribromide in pyridine (2 mL) at 0° C., and then the reaction mixture was left to stir at room temperature for 20 hours. The resulting reaction mixture was diluted with DCM (50 mL) and washed with water (50 mL), 3M HCl solution (50 mL) and saturated NaCl solution (50 mL). The organic extract was dried (Na₂SO₄) and the solvent evaporated under reduced pressure giving a white solid which after purification by flash-column chromatography (eluent; DCM:MeOH, 200:1) gave a residue containing a mixture of Compounds 5-7. Compounds 5, 6 and 7 were successfully separated by preparative HPLC obtaining 13 mg (7%), 23 mg (10%) and 2 mg (0.7%) respectively.

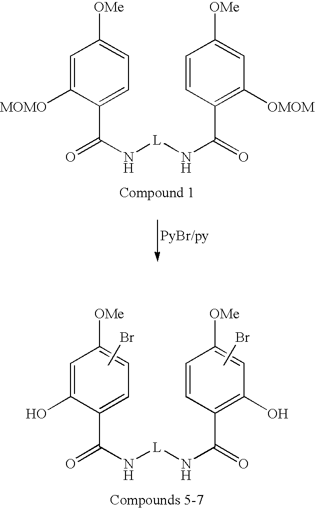

### Example 5

### Compound 5

¹H-NMR (Acetone-d₆, 400 MHz, [symbol] ppm): (8.12, brs, 2H, NH), (7.94, s, 2H), (6.54, s, 2H), (3.91, s, 6H), (3.41, m, 4H), (1.69-1.66, m, 4H), (1.49-1.47, m, 2H).

¹³C-NMR (Acetone-d₆, 100 MHz, [symbol] ppm): 169.2, 160.0, 130.6, 108.7, 101.2, 99.9, 56.1, 39.3, 29.9, 24.2.

ESI-MS[M]⁺ 561.

### Example 6

### Compound 6

¹H-NMR (CDCl₃, 400 MHz, [symbol] ppm): (7.59, s, 1H), (7.5o, s, 1H), (6.54, s, 2H), (6.20, brs, 2H, NH), (3.91, s, 3H), (3.88, s, 3H), (3.44, m, 4H), (1.69-1.66, m, 4H), (1.49-1.47, m, 2H),

¹³C-NMR (CDCl₃, 100 MHz, [symbol] ppm): 169.0, 168.2, 162.9, 160.1, 159.2, 158.5, 129.4, 128.4, 112.6, 108.7, 108.2, 106.1, 101.4, 100.6, 60.7, 56.4, 39.8, 39.4, 29.1, 28.9, 24.1.

ESI-MS[M]⁺ 640.

### Example 7

### Compound 7

¹H-NMR (CDCl₃, 400 MHz, [symbol] ppm): (7.49, s, 2H), (6.27, brs, 2H, NH), (3.85, s, 6H), (3.40, m, 4H), (1.69-1.66, m, 4H), (1.49-1.47, m, 2H).

¹³C-NMR (CDCl₃, 100 MHz, [symbol] ppm): 168.23, 1590.30, 158.7, 128, 112.6, 101.85, 106.2, 60.7, 39.7, 29.0, 24.0.

ESI-MS[M]⁺ 719.

### Examples 8-11

### Preparation of Compounds 8, 9, 10, 11

Compounds 8, 9, 10 and 11 were synthesised starting from Compound 2 according to the following general scheme:

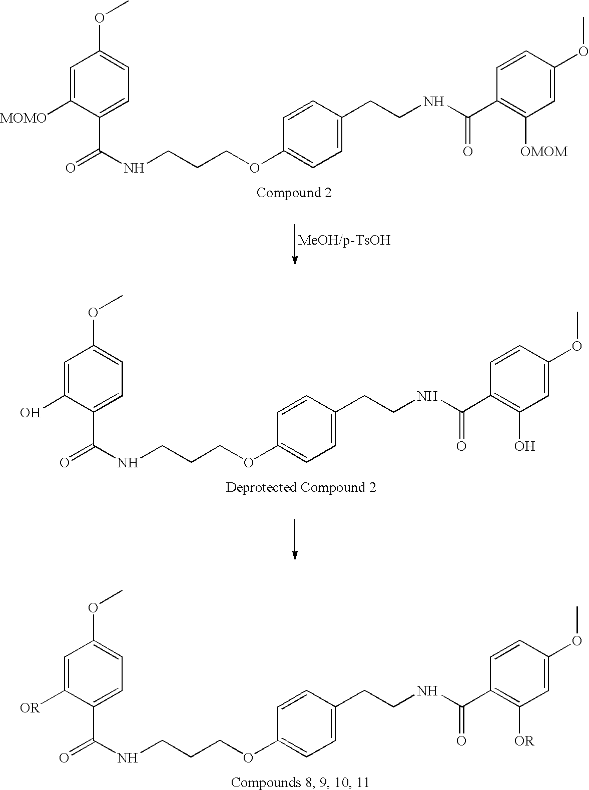

To a white suspension of Compound 2 (1.38 g, 2.37 mmol) in MeOH (35 mL), p-TsOHxH₂O (226 mg) was added and the suspension was stirred for 20 hours at room temperature. The solvent was evaporated under reduced pressure, water (25 mL) was added and the white precipitate was filtered and rinsed a few times with water to give 0.99 g (84%) of the Deprotected Compound 2 as a white solid. This compound was treated with K₂CO₃ in DMF and the resulting mixture was stirred for 45 minutes. The corresponding alkylating agent was then added and the reaction mixture was left to stir for 1 day at room temperature. After evaporating the solvent under reduced pressure, water was added (100 mL) and the resulting mixture was extracted with DCM (2×50 mL). The combined extracts were washed with a saturated NaCl solution (100 mL), were dried (Na₂SO₄), and the solvent evaporated under reduced pressure, to give a residue which was further purified as further detailed below for each case.

### Example 8

### Preparation of Compound 8

Reagents: Deprotected Compound 2 (100 mg, 0.2 mmol), potassium carbonate (110 mg, 0.8 mmol), anhydrous DMF (3 mL) and ethyl bromoacetate (0.07 mL, 0.6 mmol).

Purification: was not required.

Yield: 130 mg (93%) as white solid.

¹H-NMR (CDCl₃, 400 MHz, δ ppm): 8.51 (t, 1H, J=5.4 Hz), 8.38 (t, 1H, J=5.5 Hz), 8.22 (1H, s), 8.20 (1H, s), 7.15 (d, 2H, J=8.6 Hz), 6.82 (d, 2H, J=8.6 Hz), 6.63 (dd, 2H, J=2.2 Hz, J=8.8 Hz), 6.32 (dd, 1H, J=2.3 Hz, J=3.6 Hz), 4.61 (s, 2H), 4.60 (s, 2H), 4.28 (q, 2H, J=7.2 Hz), 4.24 (q, 2H, J=7.2 Hz), 4.05 (t, 2H, J=6.3 Hz), 3.84 (s, 3H), 3.83 (s, 3H), 3.68 (m, 4H), 2.90 (m, 2H), 2.15 (m, 2H, J=6.5 Hz), 1.30 (q, 2H, J=7.2 Hz).

¹³C-NMR (CDCl₃, 100 MHz, δ ppm): 168.1, 168.0, 689.2, 165.0, 164.8, 163.3, 163.2, 157.8, 156.9, 156.9, 134.4, 134.4, 131.8, 129.9, 115.5, 115.5, 114.6, 106.3, 99.7, 66.0, 65.8, 62.1, 62.0, 55.8, 41.7, 37.2, 35.1, 29.5, 14.4.

ESI-MS[M]⁺ 667.

### Example 9

### Preparation of Compound 9

Reagents: Deprotected Compound 2 (150 mg, 0.3 mmol), potassium carbonate (166 mg, 1.2 mmol), anhydrous DMF (4 mL) and iodopropane (0.09 mL, 0.9 mmol). Purification: was not required.

Yield: 165 mg (94%) as clear yellow solid.

¹H-NMR (CDCl₃, 400 MHz, δ ppm): 8.19 (d, 1H, J=6.2 Hz), 8.17 (d, 1H, J=6.2 Hz), 8.04 (t, 1H, J=5.3 Hz), 7.93 (t, 1H, J=5.4 Hz), 7.13 (d, 2H, J=8.5 Hz), 6.83 (d, 2H, J=8.5 Hz), 6.59 (t, 1H, J=2.5, Hz), 6.57 (t, 1H, J=2.5 Hz), 6.44 (d, 1H, J=2.3 Hz), 6.41 (d, 1H, J=2.3 Hz), 4.03 (t, 2H, J=6.1 Hz), 3.99 (t, 2H, J=6.5 Hz), 3.92 (t, 3H, J=6.6 Hz), 3.83 (s, 3H), 3.82 (s, 3H), 3.69 (dd, 2H, J=7.1 Hz, J=12.9 Hz), 3.64 (dd, 2H, J=7.2 Hz, J=13.2), 2.84 (t, 3H, J=7.0 Hz), 2.09 (p, 2H, J=6.4 Hz), 1.79 (m, 2H), 1.63 (m, 2H), 0.99 (t, 3H, J=7.4 Hz), 0.91 (t, 3H, J=7.4 Hz).

¹³C-NMR (CDCl₃, 100 MHz, δ ppm): 165.3, 165.1, 163.2, 163.1, 158.2, 157.5, 133.8, 133.7, 131.4, 129.6, 114.6, 114.5, 105.2, 105.1, 99.3, 70.5, 70.4, 65.6, 55.4, 40.9, 36.6, 34.9, 29.3, 22.4, 22.2, 10.5, 10.4.

ESI-MS[M]⁺ 579.

### Example 10

### Preparation of Compound 10

Reagents: Deprotected Compound 2 (150 mg, 0.3 mmol), potassium carbonate (165 mg, 1.2 mmol), anhydrous DMF (4 mL) and bromoethylmethyl ether (0.12 mL, 1.3 mmol). Purification: silica gel flash-column chromatography using EtOAc:MeOH (200:1).

Yield: 116 mg (63%) as white solid.

¹H-NMR (CDCl₃, 400 MHz, δ ppm): 8.16 (m, 4H), 7.14 (d, 2H, J=8.0 Hz), 6.83 (d, 2H, J=8.1 Hz), 6.61 (d, 2H, J=8.8 Hz), 6.44 (m, 2H), 4.17 (m, 1H), 4.13 (m, 4H), 4.04 (t, 2H, J=6.2 Hz), 3.83 (s, 6H), 3.69 (m, 2H), 3.62 (m, 4H), 3.36 (s, 2H), 3.34 (s, 2H), 2.85 (t, 2H, J=7.3 Hz), 2.09 (t, 2H, J=9.8 Hz).

¹³C-NMR (CDCl₃, 100 MHz, δ ppm): 165.2, 165.0, 163.0, 163.0, 158.0, 157.9, 157.4, 133.7, 131.7, 129.7, 115.2, 114.5, 105.8, 105.8, 99.9, 99.90, 70.3, 70.2, 67.9, 65.6, 58.9, 58.84, 55.5, 41.4, 36.8, 34.9, 29.3.

ESI-MS[M]⁺ 611.

### Example 11

### Preparation of Compound 11

Reagents: Deprotected Compound 2 (150 mg, 0.3 mmol), potassium carbonate (103 mg, 0.7 mmol), anhydrous DMF (4 mL) and chloroacetone (0.07 mL, 0.9 mmol). Purification: silica gel flash-column chromatography using EtOAc:MeOH (200:1).

Yield: 98 mg (53%) as white solid.

¹H-NMR (CDCl₃, 400 MHz, δ ppm): 8.63 (brs, 1H), 8.43 (brs, 1H), 8.21 (s, 1H), 8.19 (s, 1H), 7.15 (d, 2H, J=7.9 Hz), 6.80 (d, 2H, J=8.0 Hz), 6.62 (d, 2H, J=8.7 Hz), 6.29 (s, 1H), 6.27 (s, 1H), 4.66 (s, 2H), 4.63 (s, 2H), 4.06 (t, 2H, J=6.1 Hz), 3.83 (s, 3H), 3.82 (s, 3H), 3.70 (m, 4H), 2.92 (t, 2H, J=7.2 Hz), 2.19 (m, 8H, J=6.9 Hz).

¹³C-NMR (CDCl₃, 100 MHz, δ ppm): 201.2, 200.9, 164.8, 164.6, 163.0, 157.5, 156.7, 156.6, 134.2, 131.6, 129.7, 115.3, 114.4, 106.0, 105.9, 99.6, 72.9, 72.9, 65.9, 55.5, 41.4, 37.0, 34.7, 29.0, 26.0

ESI-MS[M]⁺ 607.

### Example 12

### Preparation of Compound 12

To a solution of 2,4-(dimethoxy)-3-(methyl)benzoic acid (392 mg, 2.0 mmol) in anhydrous THF (4 mL), 1,1′-carbonyldiimidazol (340 mg, 2.1 mmol) was added under N₂ atmosphere, and the resulting mixture was stirred for four hours at room temperature. Afterwards, a solution of 1,5-diaminopentane (122 mg, 1.2 mmol) and triethylamine (242 mg, 2.4 mmol) in a mixture of DMF:THF (1:1, 6 mL) was added and the reaction mixture was stirred for further 20 hours. After evaporation of the solvent under reduced pressure, water was added and the resulting mixture was extracted with DCM. The combined organic extracts were washed with saturated NaCl solution and dried with Na₂SO₄. Evaporation of the solvent under reduced pressure gave a residue which was purified by silica gel flash-column chromatography, giving Compound 12.

Purification: Silica gel flash-chromatography using EtOAc:Hexanes (1:4 to 1:1)

Yield: 102 mg (18%) as a white solid.

¹H-NMR (CDCl₃, 400 MHz, [symbol] ppm): (7.91, d, 2H, J=8.8 Hz), (7.84, brt, 2H, NH, J=5.2 Hz), (6.69, d, 2H, J=8.8 Hz), (3.84, s, 6H), (3.70, s, 6H), (3.45, q, 4H, J=5.2 Hz), (2.12, s, 6H), (1.67, m, 4H), (1.50, m, 2H).

¹³C-NMR (CDCl₃, 100 MHz, [symbol] ppm): 165.4, 161.0, 157.2, 129.8, 119.4, 118.8, 106.3, 61.3, 55.6, 39.3, 29.4, 24.5, 8.8.

ESI-MS[M]⁺ 458.7.

### Example 13

### Preparation of Compound 13

Compound 13 was prepared in two subsequent steps.

### Step 1: Preparation of the intermediate [5-(2,4,5-trimethoxy-benzoylamino)-pentyl]-carbamic acid tert-butyl ester

To a solution of 2,4,5-(trimethoxy)benzoic acid (414 mg, 2.0 mmol) in anhydrous THF (4 mL), 1,1′-carbonyldiimidazol (340 mg, 2.1 mmol) was added and the mixture was stirred under N₂ at room temperature overnight. Then a solution of 5-(aminopentyl) carbamic acid tert-butyl ester (405 mg, 2.0 mmol) and triethylamine (202 mg, 2.0 mmol) in anhydrous THF (3 mL) was added and the reaction mixture was stirred at room temperature for 20 hours. After solvent removal, the resulting residue was taken up in DCM and sequentally washed with 1N HCl, 10% K₂CO₃, water and saturated NaCl, dried with Na₂SO₄ and evaporated, providing 550 mg (69% yield) of the pure product as a colorless oil.

### Step 2: Preparation of Compound 13

[5-(2,4,5-trimethoxy-benzoylamino)-pentyl]-carbamic acid tert-butyl ester (530 mg, 1.3 mmol) was treated with a mixture of TFA:DCM (1:1, 20 mL) at room temperature for 2 hours, evaporated under reduced pressure and further dried under high vacuum over KOH for 4 hours. In a different flask, to a solution of 2,4-(dimethoxy)-3-(methyl)benzoic acid (284 mg, 1.4 mmol) in anhydrous THF (3 mL), 1,1′-carbonyldiimidazol (246 mg, 1.5 mmol) was added and the mixture was stirred under N₂ at room temperature for 6 hours. Then, a solution of the deprotected amine and triethylamine (307 mg, 3 mmol) in anhydrous THF (4 mL) was added and the reaction mixture was stirred for 48 hours at room temperature. Solvent was removed and the residue was redissolved in DCM and washed sequentally with 1N HCl, 10% K₂CO₃, water and saturated NaCl, dried with Na₂SO₄ and evaporated. Flash-chromatography on silica gel using EtOAc/DCM (1:4 to 1:1) afforded 252 mg (40% yield) of the pure product as a colorless oil.

¹H-NMR (CDCl₃, 400 MHz, [symbol] ppm): (7.84, d, 1H, J=8.8 Hz), (7.83, brt, 1H, NH, J=5.2 Hz), (7.79, brt, 2H, NH, J=5.6 Hz), (7.65, s, 1H), (6.61, d, 2H, J=8.8 Hz), (7.65, s, 1H), (3.84, s, 3H), (3.83, s, 3H), (3.80, s, 3H), (3.76, s, 3H), (3.62, s, 3H), (3.39, m, 4H), (2.04, s, 3H), (1.59, m, 4H), (1.42, m, 2H).

¹³C-NMR (CDCl₃, 100 MHz, [symbol] ppm): 165.2, 164.8, 160.8, 157.0, 152.2, 151.8, 143.0, 129.5, 119.2, 118.5, 113.8, 113.0, 106.1, 96.4, 61.1, 56.4, 56.0, 55.8, 55.4, 39.2, 39.1, 29.2, 29.1, 24.3, 8.6.

ESI-MS[M-CH₃O]⁺ 444.8

### Example 14-15

### Preparation of Compounds 14, 15

To a solution of 2,4-dimethoxy-benzoic acid in anhydrous THF, 1,1′-carbonyldiimidazol was added under N₂ atmosphere, and the resulting mixture was stirred for four hours at room temperature. Afterwards, a solution of the corresponding diamines in anhydrous THF (DMF was also added when the corresponding diamine was not soluble in THF), and TEA (2 eq, only when the diamine was used as its trifluoroacetic salt) was added and the reaction mixture was stirred for further 20 hours. After evaporation of the solvent under reduced pressure, water was added and the resulting mixture was extracted with DCM. The combined organic extracts were washed with saturated NaCl solution and dried with Na₂SO₄. Evaporation of the solvent under reduced pressure giving Compounds 14 and 15.

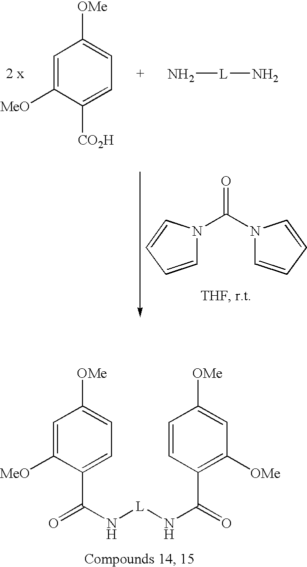

### Example 14

### Preparation of Compound 14

Reagents: 2,4-dimethoxy-benzoic acid (1000 mg, 5.5 mmol) in anhydrous THF (10 mL); 1,1′-carbonyldiimidazol (940.5 mg, 5.8 mmol); 1,6-diaminohexane (383.5 mg, 3.3 mmol) in THF (10 mL).

Yield: 943 mg (67%) as a yellow solid.

¹H-NMR (CDCl₃, 400 MHz, [symbol] ppm): (8.17, d, 2H, J=8.4 Hz), (7.75, brs, 2H, NH), (6.58, dd, 2H, J=2.4, J=8.4 Hz), (6.46, dd, 2H, J=2.4 Hz), (3.92, s, 6H), (3.84, s, 6H), (3.46-3.41, m, 4H), (1.64-1.69, m, 4H), (1.46-1.42, m, 4H).

¹³C-NMR (CDCl₃, 100 MHz, [symbol] ppm): 165.1, 163.2, 158.7.5, 133.8, 114.7, 105.2, 98.5, 55.9, 55.4, 39.5, 29.5, 26.7.

ESI-MS[M+H]⁺ 445.

### Example 15

### Preparation of Compound 15

Reagents: 2,4-dimethoxy-benzoic acid (1000 mg, 5.5 mmol) in anhydrous THF (10 mL); 1,1′-carbonyldiimidazol (940.5 mg, 5.8 mmol); 3-[4-(2-aminoethyl)-phenoxy]-propylamine diacetate salt (1392.6 mg, 3.3 mmol) [see synthesis below]; TEA (1.22 g, 12.1 mmol) and DMF 1.6 ml in THF (10 mL).

Yield: 1140 mg (67%) as a yellow solid.

¹H-NMR (CDCl₃, 400 MHz, [symbol] ppm): (8.11-8.10, brs 1H, NH), (7.98-7.95, brs, 1H, NH), (7.83-7.76, m, 2H), (7.17, d, 2H, J=8.8 Hz), (6.89, d, 2H, J=8.8 Hz), (6.61-6.60, m, 4H), (4.02-3.98, m, 2H), (3.83, s, 3H), (3.81, s, 3H), (3.80, s, 6H), (3.49-3.40, m, 4H), (2.76-2.73, m, 2H), (2.00-1.88, m, 2H).

¹³C-NMR (CDCl₃, 100 MHz, [symbol] ppm): 166.0, 165.0, 164.0, 159.7, 158.0, 133.7, 132.0, 130.0, 116.0, 115.0, 106.0, 98.5, 66.0, 56.5, 56.0, 36.5, 34.0, 29.8.

ESI-MS[M+H]⁺ 523.

### Synthesis of 3-[4-(2-aminoethyl)-phenoxy]-propylamine diacetate salt

To a solution of tyramine (4-(2-aminoethyl)-phenol) (2.0 g, 14.6 mmol) in anhydrous DCM (30 mL), TEA (4.06 mL, 29.2 mmol) was added at room temperature. BOC anhydride (1.9 g, 8.76 mmol) was slowly added at 0° C. and the resulting mixture was stirred at room temperature for 2 days. DCM (50 mL) was added and the organic phase was washed with 0.1 M HCl (50 mL), water (3×100 mL), saturated NaCl solution, and subsequently dried (Na₂SO₄), and the solvent evaporated under reduced pressure, providing 2.15 g (61%) of [2-(4-hydroxy-phenyl)-ethyl]-carbamic acid tert-butyl ester.

A mixture of the above tert-butyl ester derivative (15.6 g, 66.0 mmol), N-(3-bromopropyl)-phtalimide (12.7 g, 66.0 mmol), K₂CO₃ (22.8 g, 132 mmol) and KI (3.29 g, 19.8 mmol) in acetonitrile was refluxed for 24 hours. The solvent was evaporated to dryness, water was added (300 mL), and the resulting mixture was extracted with DCM (2×300 mL). The combined extracts were washed with saturated NaCl solution, dried (Na₂SO₄) and the solvent removed. The resulting product was triturated in acetonitrile and filtered to give 20.2 g (72%) of (2-{4-[3-(1,3-dioxo-1,3-dihydroisoindole-2-yl)propoxy]-phenyl-ethyl)-carbamic acid tert-butyl ester.

A mixture of the above compound (20.2 g, 48 mmol) with hydrazine monohydrate (6.8 mL, 140 mmol) in MeOH (400) was refluxed for 4 hours. After evaporation of the solvent, the white solid obtained was suspended in DCM and the mixture cooled in an ice-bath. Filtration of the white precipitate gave 11.53 g (86%) of 3-[4-(2-amino-propoxy)-phenylamino]-propionic acid tert-butyl ester.

Treatment of this compound (1.9 g, 7.0 mmol) with TFA (25 mL) in THF (75 mL) at room temperature for 24 hours gave 2.80 g (93%) of 3-[4-(2-aminoethyl)-phenoxy]-propylamine as diacetate salt.

### Examples 16-19

### Preparation of Compounds 16, 17, 18, 19

Compounds 16, 17, 18, 19 were synthesised starting from Compound 2 according to the following general scheme:

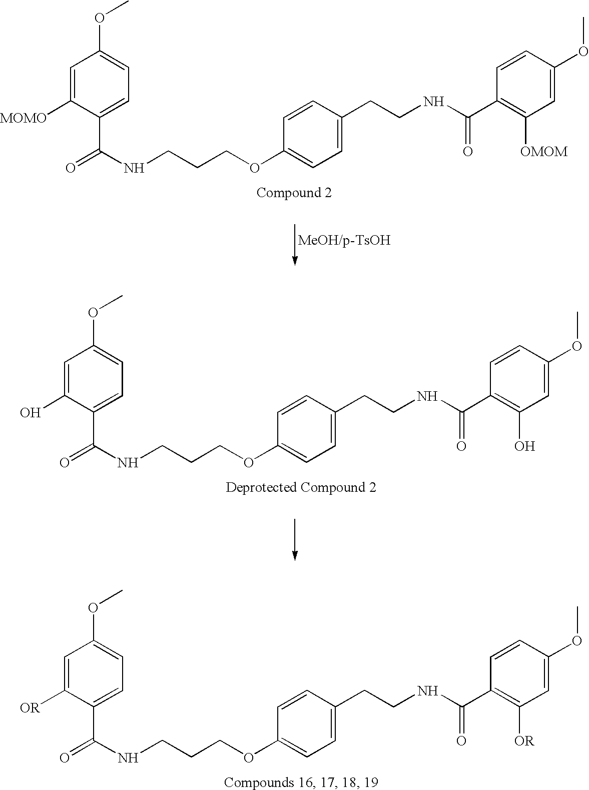

To a white suspension of Compound 2 (1.38 g, 2.37 mmol) in MeOH (35 mL), p-TsOHxH₂O (226 mg) was added and the suspension was stirred for 20 hours at room temperature. The solvent was evaporated under reduced pressure, water (25 mL) was added and the white precipitate was filtered and rinsed a few times with water to give 0.99 g (84%) of the Deprotected Compound 2 derivative as a white solid. This compound was treated with K₂CO₃ in DMF and the resulting mixture was stirred for 45 minutes. The corresponding alkylating agent was then added and the reaction mixture was left to stir for 1 day at room temperature in the case of Compounds 17 and 18, and for 3 hours at 70° C. in the case of Compounds 16 and 19. After evaporating the solvent under reduced pressure, water was added (100 mL) and the resulting mixture was extracted with DCM (2×50 mL). The combined extracts were washed with a saturated NaCl solution (100 mL), were dried (Na₂SO₄), and the solvent evaporated under reduced pressure, to give a residue which was further purified as further detailed below for each case.

### Example 16

### Preparation of Compound 16

Reagents: Deprotected Compound 2 (150 mg, 0.3 mmol), potassium carbonate (165 mg, 1.2 mmol), anhydrous DMF (4 mL) and 1-iodo-2-methylpropane (0.3 mL, 2.7 mmol). Purification: silica gel flash-column chromatography using EtOAc:MeOH (200:1).

Yield: 132 mg (72%) as white solid.

¹H-NMR (CDCl₃, 400 MHz, δ ppm): 8.20 (d, 1H, J=6.5 Hz), 8.17 (d, 1H, J=6.4 Hz), 8.04 (t, 1H, J=5.4 Hz), 7.94 (t, 1H, J=5.5 Hz), 7.13 (d, 2H, J=8.6 Hz), 6.82 (d, 2H, J=8.6 Hz), 6.58 (d, 1H, J=9.8 Hz), 6.44 (d, 2H, J=2.3 Hz), 6.41 (d, 2H, J=2.3 Hz), 4.02 (t, 2H, J=6.2 Hz), 3.84 (s, 3H), 3.83 (s, 3H), 3.82 (d, 2H, J=6.5 Hz), 3.75 (d, 2H, J=6.5 Hz), 3.66 (m, 4H), 2.85 (t, 2H, J=7.2 Hz), 2.08 (m, 3H), 1.90 (td, 1H, J=6.7 Hz, J=13.3 Hz), 1.00 (m, 3H), 0.98 (s, 3H), 0.93 (s, 3H), 0.92 (s, 3H).

¹³C-NMR (CDCl₃, 100 MHz, δ ppm): 165.4, 165.2, 163.2, 163.1, 158.3, 157.5, 133.8, 131.4, 129.6, 114.5, 114.5, 105.2, 105.1, 99.3, 75.3, 65.5, 55.5, 41.1, 36.6, 35.0, 29.3, 28.2, 28.0, 19.3, 19.2.

ESI-MS[M]⁺ 607.

### Example 17

### Preparation of Compound 17

Reagents: Deprotected Compound 2 (150 mg, 0.3 mmol), potassium carbonate (165 mg, 1.2 mmol), anhydrous DMF (4 mL) and 3,3-dimethylallylbromide (0.18 mL, 1.8 mmol).

Purification: silica gel flash-column chromatography using hexane:EtOAc (1:1).

Yield: 140 mg (73%) as white solid.

¹H-NMR (CDCl₃, 400 MHz, δ ppm): 8.19 (d, 1H, J=3.5 Hz), 8.17 (d, 1H, J=3.5 Hz), 8.08 (t, 1H, J=5.3 Hz), 7.98 (t, 1H, J=5.2 Hz), 7.13 (d, 2H, J=8.6 Hz), 6.83 (d, 2H, J=8.6 Hz), 6.59 (dd, 1H, J=2.4 Hz, J=3.3 Hz), 6.57 (dd, 1H, J=2.5 Hz, J=3.4 Hz), 6.47 (d, 1H, J=2.3 Hz), 6.43 (d, 1H, J=2.4 Hz), 5.43 (m, 1H), 5.29 (dt, 1H, J=2.0 Hz, J=6.7 Hz), 4.56 (d, 2H, J=6.9 Hz), 4.51 (d, 2H, J=6.8 Hz), 4.02 (t, 2H, J=6.2 Hz), 3.84 (s, 3H), 3.83 (s, 3H), 3.66 (dd, 2H, J=5.9 Hz, J=11.6 Hz), 3.61 (dd, 2H, J=5.6 Hz, J=11.3 Hz), 2.82 (t, 2H, J=7.1 Hz), 2.06 (p, 2H, J=6.5 Hz), 1.76 (s, 3H), 1.71 (s, 3H), 1.69 (s, 3H).

¹³C-NMR (CDCl₃, 100 MHz, δ ppm): 165.3, 165.1, 163.1, 163.0, 158.2, 158.1, 157.4, 140.0, 139.4, 133.7, 131.6, 129.7, 118.4, 118.2, 114.8, 114.5, 105.3, 105.2, 99.7, 65.8, 65.7, 65.6, 55.5, 41.1, 36.6, 34.9, 29.2, 25.7, 25.6, 18.22.

ESI-MS[M]⁺ 631.

### Example 18

### Preparation of Compound 18

Reagents: Deprotected Compound 2 (150 mg, 0.3 mmol), potassium carbonate (165 mg, 1.2 mmol), anhydrous DMF (4 mL) and 1-iodobutane (0.1 mL, 0.9 mmol).

Purification: silica gel flash-column chromatography using hexane:EtOAc (1:1).

Yield: 70 mg (38%) as white solid.

¹H-NMR (CDCl₃, 400 MHz, δ ppm): 8.18 (t, 2H, J=7.8 z), 8.04 (m, 1H), 7.94 (t, 1H, J=5.1 Hz), 7.13 (d, 2H, J=8.1 Hz), 6.83 (d, 2H, J=7.9 Hz), 6.58 (d, 2H, J=8.6 Hz), 6.44 (d, 2H, J=13.2 Hz), 4.04 (dd, 4H, J=5.5 Hz, J=9.8 Hz), 3.97 (t, 2H, J=6.5 Hz), 3.84 (m, 6H), 3.66 (m, 4H), 2.84 (t, 2H, J=6.9 Hz), 2.09 (m, 2H), 1.76 (m, 2H), 1.60 (m, 2H), 1.44 (dd, 2H, J=7.3 Hz, J=14.9 Hz), 1.35 (dd, 2H, J=7.9 Hz, J=14.3 Hz), 0.92 (t, 6H, J=7.3 Hz).

¹³C-NMR (CDCl₃, 100 MHz, δ ppm):

ESI-MS[M]⁺ 607.

### Example 19

### Preparation of Compound 19

Reagents: Deprotected Compound 2 (100 mg, 0.2 mmol), potassium carbonate (110 mg, 0.8 mmol), anhydrous DMF (3 mL) and 2-bromobutane (0.09 mL, 0.9 mmol).

Purification: silica gel flash-column chromatography using hexane:EtOAc (1:1).

Yield: 95 mg (78%) as white solid.

¹H-NMR (CDCl₃, 400 MHz, δ ppm): 8.19 (d, 1H, J=5.5 Hz), 8.17 (d, 1H, J=5.5 Hz), 8.12 (t, 1H, J=5.5 Hz), 8.01 (t, 1H, J=5.4 Hz), 7.13 (d, 2H, J=8.6 Hz), 6.83 (d, 2H, J=6.7 Hz), 6.56 (m, 2H), 6.44 (d, 1H, J=2.3 Hz), 6.40 (d, 1H, J=2.3 Hz), 4.42 (dd, 1H, J=6.0 Hz, J=12.1 Hz), 4.35 (dd, 1H, J=6.1 Hz, J=12.1 Hz), 4.02 (t, 2H, J=6.2 Hz), 3.82 (s, 3H), 3.81 (s, 3H), 3.70 (dd, 2H, J=6.5 Hz, J=13.4 Hz), 3.62 (m, 2H), 2.84 (t, 2H, J=7.0 Hz), 2.08 (p, 2H, J=6.5 Hz), 1.61 (m, 4H), 1.29 (d, 3H, J=6.1 Hz), 1.19 (d, 3H, J=6.1 Hz), 0.93 (t, 3H, J=7.5 Hz), 0.86 (t, 3H, J=7.5 Hz).

¹³C-NMR (CDCl₃, 100 MHz, δ ppm): 165.7, 165.5, 163.3, 163.2, 157.7, 157.5, 134.1, 131.7, 129.8, 115.6, 114.8, 105.5, 105.4, 100.9, 100.7, 65.7, 55.7, 41.0, 36.7, 35.1, 29.5, 29.33, 29.10, 19.40, 19.22, 9.88.

ESI-MS[M]⁺ 607.

### Example 20

### Preparation of Compound 20

Compound 20 was prepared in three subsequent steps:

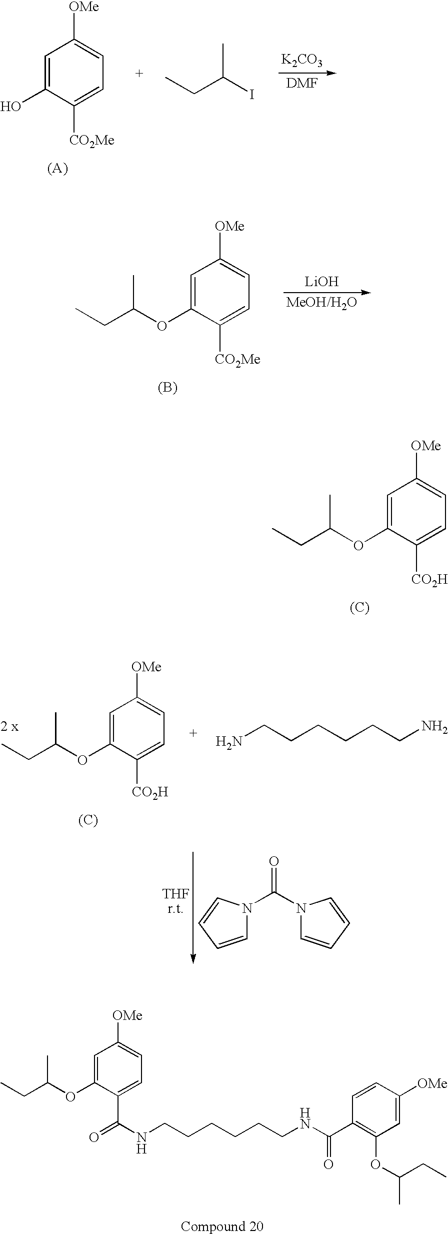

### Step 1: Synthesis of 2-sec-Butoxy-4-methoxy-benzoic acid methyl ester (B)

A mixture of 2-hydroxy-4-methoxy-benzoic acid methyl ester (A) (0.461 g, 2.53 mmol) in DMF anhydrous (17 mL) and K₂CO₃ (0.7 g, 5.07 mmol) under N₂ atmosphere was stirring at room temperature for 1 hour. Afterwards, 2-bromopropane (0.41 mL, 3.80 mmol) is added and the mixture is stirred at 70° C. for 20 hours.

After evaporation of the solvent to reduced pressure, ethyl acetate (100 mL) was added and the solution was washed with water (100 mL). The combined organic extracts were washed with saturated NaCl solution and dried with Na₂SO₄. Evaporation of the solvent under reduced pressure gave 0.30 g (50%) of 2-sec-Butoxy-4-methoxy-benzoic acid methyl ester (B) as a white solid.

Purification: was not required.

Yield: 0.30 g (50%) as a liquid.

¹H-NMR (CDCl₃, 400 MHz, [symbol] ppm): 7.82 (dd, J=7.78, 1.26 Hz, 1H), 6.49 (d, J=7.79 Hz, 1H), 6.47 (d, J=1.13 Hz, 1H), 4.33 (sext., J=6.02 Hz, 1H), 3.84 (s, 3H), 3.83 (s, 3H), 1.94-1.59 (m, 2H), 1.33 (d, J=6.09 Hz, 3H), 1.00 (t, J=7.45 Hz, 3H)

¹³C-NMR (CDCl₃, 100 MHz, [symbol] ppm): 166.46, 163.86, 159.99, 133.73, 113.80, 104.71, 101.60, 76.59, 55.41, 51.50, 29.18, 19.03, 9.59.

ESI-MS[M⁺-CH₃] 223.81

### Step 2: Synthesis of the intermediate 2-sec-Butoxy-4-methoxy-benzoic acid (C)

A mixture of 2-sec-Butoxy-4-methoxy-benzoic acid methyl ester (B) (0.11 g, 0.42 mmol) was hydrolysed by treatment with lithium hydroxide monohydrate (0.18 g, 4.20 mmol) in water/MeOH 1:1 (10 mL) for 24 hours. The water phase cooled in an ice-bath, was neutralised to pH 3-4 with 0.1 M HCl solution, and extracted with ethyl acetate (4×25 mL). The combined extracts were dried (Na₂SO₄) and the solvent evaporated, to give 0.09 g (92%) of 2-sec-Butoxy-4-methoxy-benzoic acid (C) as a white solid.

Purification: was not required.

Yield: 0.09 g (92%) as white solid.

¹H-NMR (CDCl₃, 400 MHz, δ ppm): 10.97 (s, 1H), 8.15 (d, J=8.84 Hz, 1H), 6.64 (dd, J=8.84, 2.31 Hz, 1H), 6.51 (d, J=2.29 Hz, 1H), 4.60 (sext, J=6.03 Hz, 1H), 3.87 (s, 3H), 1.96-1.70 (m, 12H), 1.43 (d, J=6.14 Hz, 3H), 1.03 (t, J=7.48 Hz, 3H)

¹³C-NMR (CDCl₃, 100 MHz, [symbol] ppm): 165.37, 164.86, 157.97, 135.56, 111.40, 106.62, 100.68, 78.83, 55.69, 28.99, 19.16, 9.58.

ESI-MS[M]⁺ 225

### Step 3: Synthesis of Compound 20

To a solution of 2-sec-Butoxy-4-methoxy-benzoic acid (C) (81 mg, 0.391 mmol) in anhydrous THF (10 mL), 1,1′-carbonyldiimidazol (66.4 mg, 0.410 mmol) was added under N₂ atmosphere, and the resulting mixture was stirred for four hours at room temperature. Afterwards, a solution of 1,6-diaminohexane (27.14 mg, 0.234 mmol) in anhydrous THF (4 mL) was added and the reaction mixture was stirred for further 20 hours. After evaporation of the solvent under reduced pressure, water was added and the resulting mixture was extracted with DCM. The combined organic extracts were washed with saturated NaCl solution and dried with Na₂SO₄. Evaporation of the solvent under reduced pressure gave a residue which was purified by silica gel flash-column chromatography.

Purification: silica gel flash-chromatography using EtOAc:Hex (1:1).

Yield: 39 mg (19%) as a white solid.

¹H-NMR (CDCl₃, 400 MHz, [symbol] ppm): (8.16, d, 2H, J=8.8 Hz), (7.98, brs, 2H, NH), (6.55, d, 2H, J=8.8 Hz), (6.43, brs, 2H), (4.46, sex, 4H, J=6.0 Hz), (3.81, s, 6H), (3.42, m, 4H), (1.79-1.70, m, 4H) (1.59, brs, 4H), (1.43, brs, 4H), (1.34, d, 6H, J=6.0 Hz), (0.99, t, 6H, J=7.4 Hz)

¹³C-NMR (CDCl₃, 100 MHz, [symbol] ppm): 165.2, 162.9, 157.2, 133.8, 115.5, 105.1, 100.5, 100.5, 76.6, 55.4, 39.4, 29.6, 29.1, 27.0, 19.2, 9.7.

ESI-MS[M]⁺=529.

### Example 21

### Preparation of Compound 21

Compound 21 was prepared in three subsequent steps:

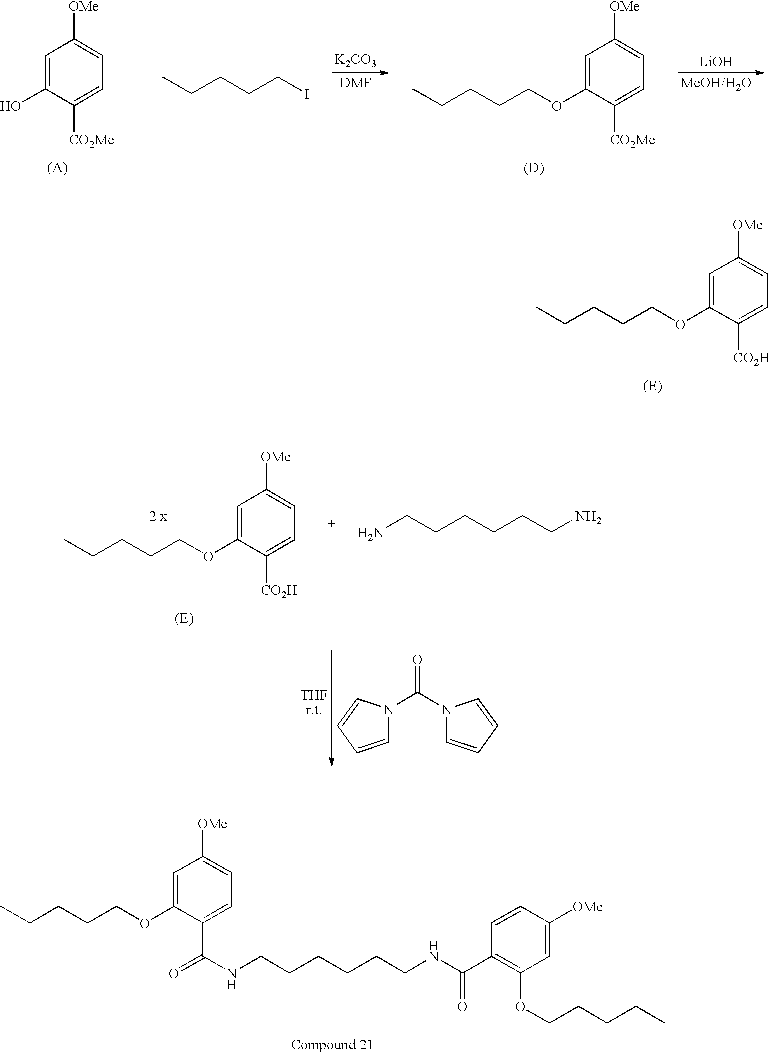

### Step 1: Synthesis of 4-Methoxy-2-pentyloxy-benzoic acid methyl ester (D)

A mixture of 2-hydroxy-4-methoxy-benzoic acid methyl ester (A) (1.5 g, 8.23 mmol) in DMF anhydrous (25 mL) and K₂CO₃ (2.2 g, 16.5 mmol) under N₂ atmosphere was stirring at room temperature for 1 hour. Afterwards, iodopentane (2.44 g, 12.3 mmol) is added and the mixture is stirred for 20 hours.

After evaporation of the solvent to reduced pressure, ethyl acetate (100 mL) was added and the solution was washed with water (100 mL). The combined organic extracts were washed with saturated NaCl solution and dried with Na₂SO₄. Evaporation of the solvent under reduced pressure gave 1.62 g (78%) of 4-Methoxy-2-pentyloxy-benzoic acid methyl ester (D) as a yellowish solid.

Purification: was not required.

Yield: 1.62 g (78%) as a yellowish solid.

¹H-NMR (CDCl₃, 400 MHz, [symbol] ppm): 7.81 (d, 1H, J=8.4 Hz), 6.46 (m, 1H), 6.43 (s, 1H), 3.97 (t, 2H, J=6.5 Hz), 3.83 (s, 3H), 3.80 (s, 3H), 1.82 (m, 2H), 1.46 (m, 2H), 1.38 (m, 2H), 0.92 (t, 3H, J=7.2 Hz).

¹³C-NMR (CDCl₃, 100 MHz, [symbol] ppm): 166.5, 164.3, 161.0, 133.9, 112.8, 104.7, 100.0, 69.1, 55.6, 51.7, 29, 28.3, 22.6, 14.2.

ESI-MS[M+H]⁺ 252.9

### Step 2: Synthesis of 4-Methoxy-2-pentyloxy-benzoic acid (E)

A mixture of 4-Methoxy-2-pentyloxy-benzoic acid methyl ester (D) (1.5 g, 6.0 mmol) was hydrolysed by treatment with lithium hydroxide monohydrate (2.5 g, 60.0 mmol) in water/MeOH/THF 1:1:1 (30 mL) for 15 hours. THF was evaporated and the water phase cooled in an ice-bath, was neutralised to pH 3-4 with 0.1 M HCl solution, and extracted with ethyl acetate (4×50 mL). The combined extracts were dried (Na₂SO₄) and the solvent evaporated, to give 1.24 g (88%) of 4-Methoxy-2-pentyloxy-benzoic acid (E) as a white solid.

Purification: was not required.

Yield: 1.24 g (88%) as white solid.

¹H-NMR (CDCl₃, 400 MHz, δ ppm): 8.02 (d, 1H, J=8.8 Hz), 6.54 (d, 1H, J=8.9 Hz), 6.44 (s, 1H), 4.05 (t, 2H, J=6.5 Hz), 3.80 (s, 3H), 1.82 (m, 2H), 1.40 (m, 4H), 0.92 (t, 3H, J=7.2 Hz).

¹³C-NMR (CDCl₃, 100 MHz, [symbol] ppm): 166.0, 165.2, 159.4, 135.5, 110.6, 106.7, 99.5, 70.3, 55.9, 28.7, 28.1, 22.4, 14.0.

ESI-MS[M]⁺ 238.8

### Step 3: Synthesis of Compound 21

To a solution of 4-Methoxy-2-pentyloxy-benzoic acid (E) (0.5 g, 2.0 mmol) in anhydrous THF (20 mL), 1,1′-carbonyldiimidazol (0.38 g, 2.20 mmol) was added under N₂ atmosphere, and the resulting mixture was stirred for four hours at room temperature. Afterwards, a solution of the corresponding 1,6-diaminohexane (0.15 g, 1.26 mmol) in anhydrous THF (5 mL) was added and the reaction mixture was stirred for further 20 hours. After evaporation of the solvent under reduced pressure, water was added and the resulting mixture was extracted with DCM. The combined organic extracts were washed with saturated NaCl solution and dried with Na₂SO₄. Evaporation of the solvent under reduced pressure gave a residue which was purified by silica gel flash-column chromatography MeOH/ethyl acetate (1:100), giving 0.241 g (22%) of Compound 21 as a white solid.

Purification: silica gel flash-chromatography using EtOAc:MeOH (100:1).

Yield: 241 mg (22%) as a white solid.

¹H-NMR (CDCl₃, 400 MHz, [symbol] ppm): 8.11 (d, 2H, J=8.8 Hz), 7.84-7.82 (m, 2H, br), 6.51 (dd, 2H, J=2.3 Hz, J=8.8 Hz), 6.38 (d, 2H, J=2.3 Hz), 4.00 (t, 2H, J=6.5 Hz), 3.76 (s, 6H, CH3), 3.37 (dd, 2H, J=7.0 Hz, J=12.6 Hz), 1.86-1.77 (m, 4H), 1.55 (m, 4H), 1.42-1.30 (m, 4H), 0.86 (t, 3H, J=7.11 Hz).

¹³C-NMR (CDCl₃, 100 MHz, [symbol] ppm): 165.3, 163.3, 158.8, 134.0, 114.9, 105.3, 99.4, 69.1, 55.6, 39.7, 29.8, 29.1, 28.5, 27.1, 22.46.

ESI-MS[M]⁺ 557.08

### Example 22

### Preparation of Compound 22

Compound 22 was prepared in three subsequent steps:

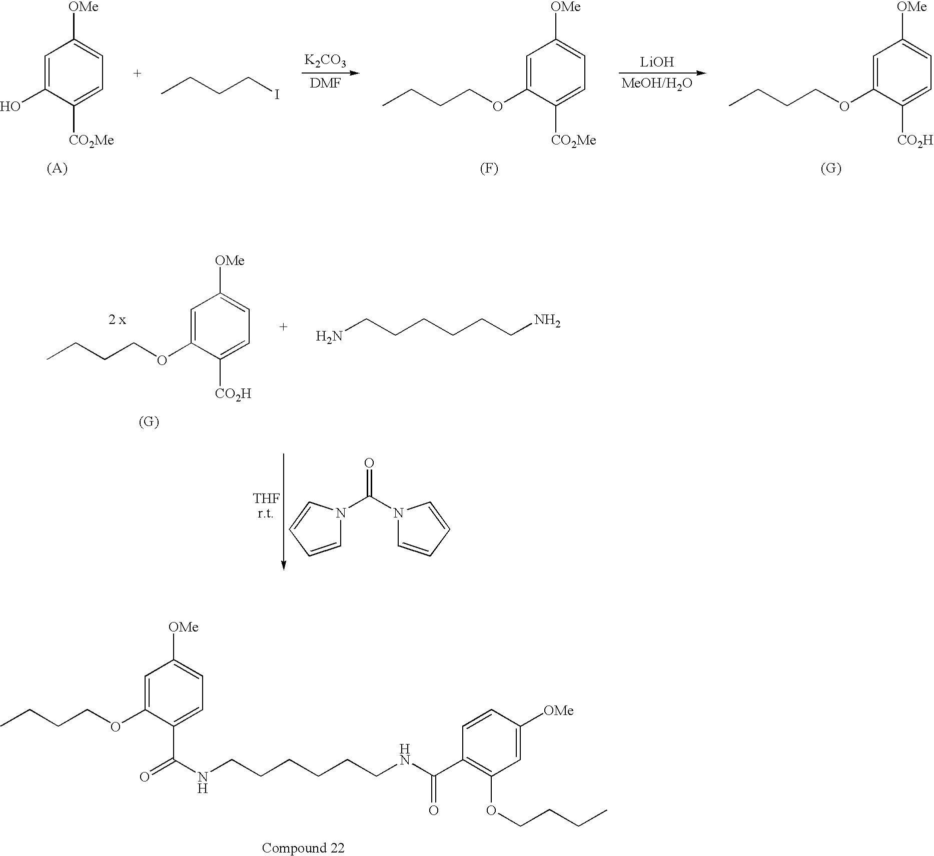

### Step 1: Synthesis of 2-Butoxy-4-methoxy-benzoic acid methyl ester (F)

A mixture of 2-hydroxy-4-methoxy-benzoic acid methyl ester (A) (0.461 g, 2.53 mmol) in DMF anhydrous (17 mL) and K₂CO₃ (0.7 g, 5.07 mmol) under N₂ atmosphere was stirring at room temperature for 1 hour. Afterwards, 1-iodobutane (0.43 mL, 3.80 mmol) is added and the mixture is stirred for 20 hours.

After evaporation of the solvent to reduced pressure, ethyl acetate (100 mL) was added and the solution was washed with water (100 mL). The combined organic extracts were washed with saturated NaCl solution and dried with Na₂SO₄. Evaporation of the solvent under reduced pressure gave 0.47 g (78%) of 2-Butoxy-4-methoxy-benzoic acid methyl ester (F) as a white solid.

Purification: was not required.

Yield: 0.47 g (78%) as a white solid.

¹H-NMR (CDCl₃, 400 MHz, [symbol] ppm): 7.84 (dd, J=8.34, 0.58 Hz, 1H), 6.50-6.45 (m, 2H, H2), 4.01 (t, J=6.49, 6.49 Hz, 2H), 3.85 (s, 3H), 3.84 (s, 3H), 1.87-1.77 (m, 2H), 1.54 (qd, J=14.75, 7.37 Hz, 2H), 0.98 (t, J=7.40 Hz, 3H)

¹³C-NMR (CDCl₃, 100 MHz, [symbol] ppm): 166.33, 164.07, 160.84, 133.76, 112.62, 104.54, 99.86, 68.59, 55.42, 51.56, 31.15, 19.18, 13.81

ESI-MS[M+H]⁺ 239

### Step 2: Synthesis of 2-Butoxy-4-methoxy-benzoic acid (G)

A mixture of 2-Butoxy-4-methoxy-benzoic acid methyl ester (F) (0.39 g, 1.64 mmol) was hydrolysed by treatment with lithium hydroxide monohydrate (069 g, 10.00 mmol) in water/MeOH 1:1 (14 mL) for 24 hours. The water phase cooled in an ice-bath, was neutralised to pH 3-4 with 0.1 M HCl solution, and extracted with ethyl acetate (4×25 mL). The combined extracts were dried (Na₂SO₄) and the solvent evaporated, to give 0.33 g (90%) of 2-Butoxy-4-methoxy-benzoic acid (G) as a liquid.

Purification: was not required.

Yield: 0.33 g (90%) as liquid.

¹H-NMR (CDCl₃, 400 MHz, δ ppm): 8.12 (d, J=8.81 Hz, 1H), 6.63 (dd, J=8.82, 2.30 Hz, 1H), 6.51 (d, J=2.30 Hz, 1H), 4.21 (t, J=6.55 Hz, 2H), 3.86 (s, 3H), 1.94-1.84 (m, 2H), 1.52 (ddt, J=14.78, 8.43, 6.63 Hz, 2H), 1.00 (t, J=7.39 Hz, 3H)

¹³C-NMR (CDCl₃, 100 MHz, [symbol] ppm): 165.2, 164.98, 158.91, 135.45, 110.46, 106.56, 99.38, 69.88, 55.70, 30.79, 19.10, 13.63.

ESI-MS[M]⁺ 225

### Step 3: Synthesis of Compound 22

To a solution of 2-Butoxy-4-methoxy-benzoic acid (G) (0.15 g, 0.7 mmol) in anhydrous THF (7 mL), 1,1′-carbonyldiimidazol (0.11 g, 0.70 mmol) was added under N₂ atmosphere, and the resulting mixture was stirred for 16 hours at room temperature. Afterwards, a solution of the corresponding 1,6-diaminohexane (0.047 g, 0.40 mmol) in anhydrous THF (2 mL) was added and the reaction mixture was stirred for further 20 hours. After evaporation of the solvent under reduced pressure, water was added and the resulting mixture was extracted with DCM. The combined organic extracts were washed with saturated NaCl solution and dried with Na₂SO₄. Evaporation of the solvent under reduced pressure gave a residue which was purified by silica gel flash-column chromatography MeOH/ethyl acetate (1:100), giving 0.20 g (54%) of Compound 22 as a white solid.

Purification: silica gel flash-chromatography using EtOAc:MeOH (100:1).

Yield: 200 mg (54%) as a white solid.

¹H-NMR (CDCl₃, 400 MHz, [symbol] ppm): 7.91 (d; J=8.66 Hz; 1H; H5); 6.62 (dd; J=8.67; 2.34 Hz; 1H); 6.59 (d; J=2.27 Hz; 1H); 4.13 (t; J=6.36 Hz; 2H); 3.84 (s; 3H); 3.42 (t; J=6.86 Hz; 2H); 1.90-1.80 (m; 2H); 1.65 (p; J=6.69 Hz; 2H); 1.58-1.45 (m; 4H; H2); 1.00 (t; J=7.40 Hz; 3H)

¹³C-NMR (CDCl₃, 100 MHz, [symbol] ppm): 167.90; 165.25; 160.08; 133.89; 115.31; 106.80; 100.27; 70.08; 56.08; 40.63; 32.42; 30.55; 28.02; 20.58; 14.22.

ESI-MS[M]⁺ 528.6

### Biological Activity of the Compounds of Formula (I)

### Example 23

### VDCC Inhibition of the Compounds

This assay is aimed to determine the VDCC blocker activity of compounds; it is performed using SH-SY5Y neuroblastoma cells. SH-SY5Y cells were plated at 5×10⁴ cells per well into Black/Clear Bottom 96-well culture plate, 48 hours before treatment. Cells were loaded with Fluo-4, 5 μM and pluronic acid, 0.1%, for 30 min at 37° C., 5% CO₂, following a incubation of 15 min at RT in Krebs-HEPES solution. Immediately cells are exposed to the samples for 10 min at different concentrations, depending on potency. After the treatment, calcium entry is measured as fluorescence in a Fluostar Optima plate reader (BMG) in response to depolarization with 60 mM KCl. The excitation wavelength was 485 nm, and that of emission 520 nm.

The compounds of formula (I) according to the present invention showed VDCC blocking activity. Results are shown in table 2.

TABLE 2

| Compound | |
|---|---|
| No. | Structure |
| 1 | 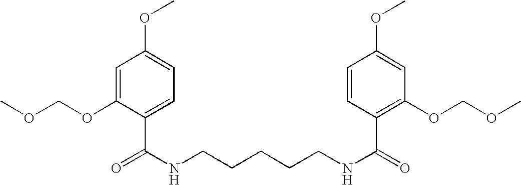 |
| 2 | 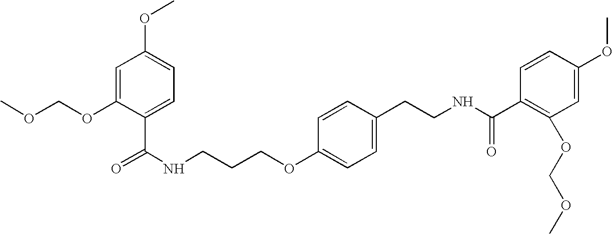 |
| 3 | 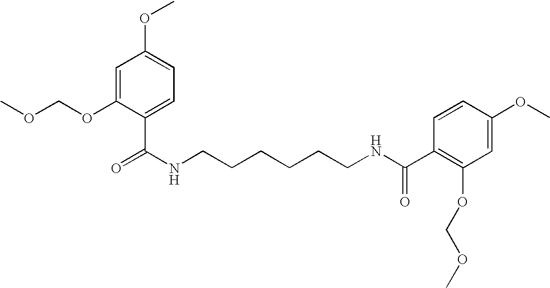 |
| 4 | 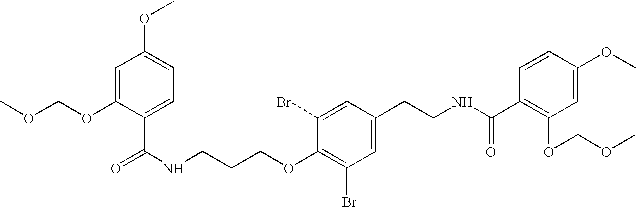 |
| 5 | 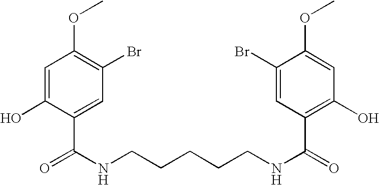 |
| 6 | 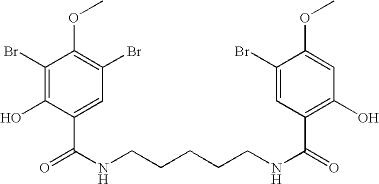 |
| 7 | 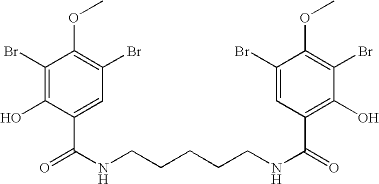 |
| 8 | 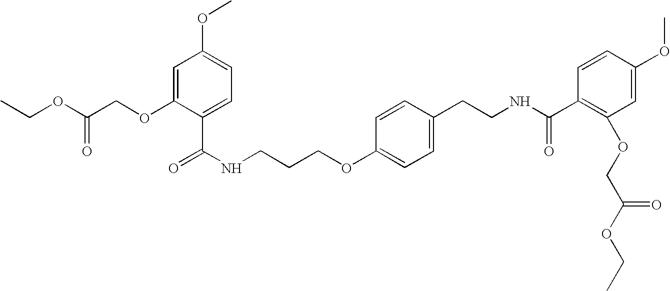 |
| 9 | 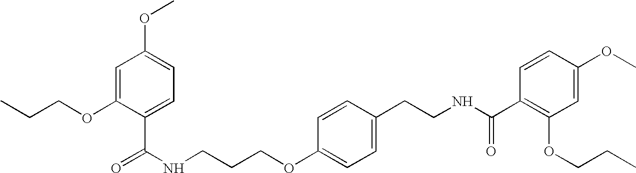 |
| 10 | 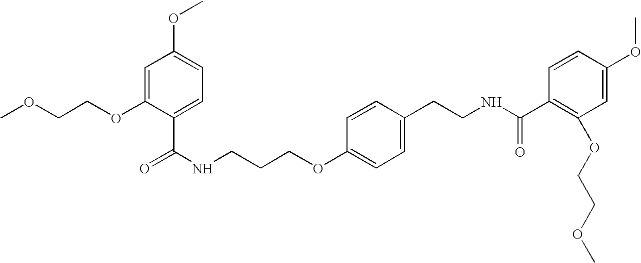 |
| 11 | 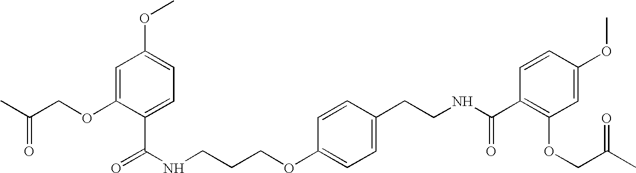 |
| 12 | 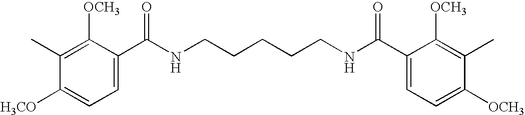 |
| 13 | 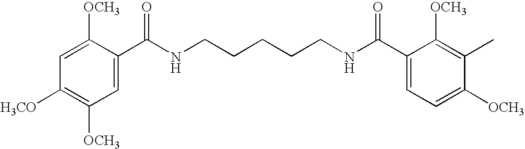 |
| 14 | 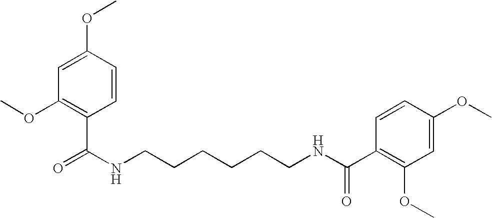 |
| 15 | 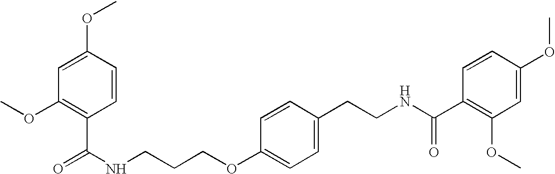 |
| 16 | 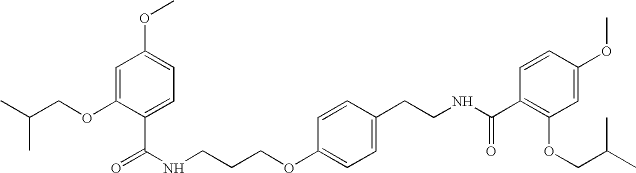 |
| 17 | 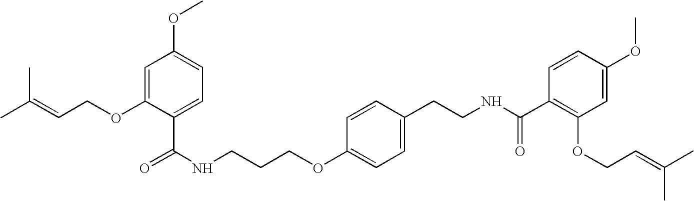 |
| 18 | 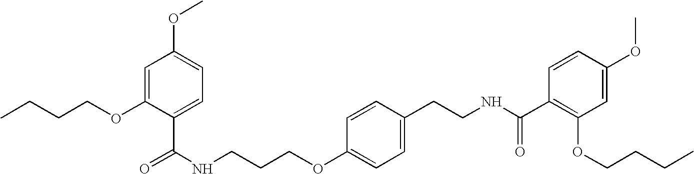 |
| 19 | 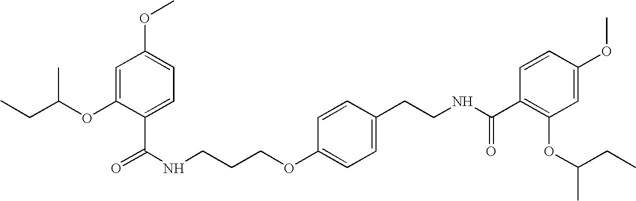 |
| 20 | 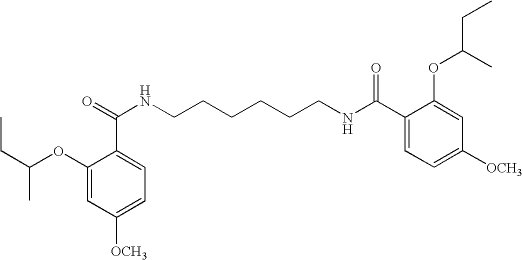 |
| 21 | 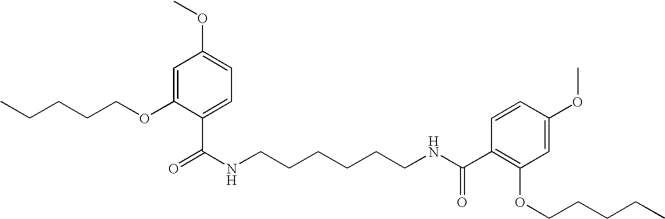 |
| 22 | 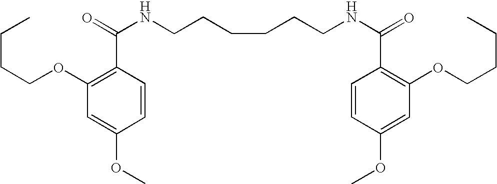 |

| | % of calcium entry | | | |
|---|---|---|---|---|
| Compound | inhibition | | | |
| No. | 10⁻⁵ | 10⁻⁶ | 10⁻⁷ | 10⁻⁸ |
| 1 | 57.8 ± 16 | / | / | / |
| 2 | 93.3 ± 5 | 34.2 ± 1.5 | 29.7 ± 14 | 31.2 ± 9 |
| 3 | 37.5 ± 7 | 6.6 ± 2 | / | / |
| 4 | 87.3 ± 5 | 27.3 ± 5 | 6 ± 0 | / |
| 5 | 21.8 ± 5 | 0 | / | / |
| 6 | 96.6 ± 4 | 48.7 ± 18 | 27.5 ± 5 | 11.8 ± 16 |
| 7 | 90.7 ± 3 | 3.1 ± 0 | / | / |
| 8 | 57 ± 7 | 0 | / | / |
| 9 | 86 ± 0.5 | 61 ± 0 | 0 | / |
| 10 | 79 ± 0.1 | 22 ± 19 | 0 | / |
| 11 | 36 ± 3 | 0 | / | / |
| 12 | 78 ± 2.5 | 0 | / | / |
| 13 | 27 ± 2 | 0 | / | / |
| 14 | 38 ± 6 | 0 | / | / |
| 15 | 91.5 ± 1.5 | 37 ± 5 | 0 | / |
| 16 | 39 ± 4 | 0 | / | / |
| 17 | 45 ± 12 | 39 ± 8 | 0 | / |
| 18 | 37 ± 2 | 0 | / | / |
| 19 | 75 ± 12 | 69 ± 1 | 0 | / |
| 20 | 91 ± 3 | 59 ± 19 | 0 | / |
| 21 | 27 ± 7 | 0 | / | / |
| 22 | 24 ± 12 | 0 | / | / |

### Example 24

### Toxicity Measurement

The cytotoxicity effect of the molecules was tested in the human neuroblastoma cell line SH-SY5Y. These cells were cultured in 96-well plates in minimum essential medium, Ham's F12 medium, supplemented with 10% fetal bovine serum, 1% glutamine and 1% penicillin/streptomycin, and grown in a 5% CO₂ humidified incubator at 37° C. Cells were plated at 10⁴ cells for each well, at least, 48 hours before treatment. Cells were exposed for 24 hours to the compounds at different concentrations, quantitative assessment of cell death was made by measurement of the intracellular enzyme lactate dehydrogenase (LDH) (citotoxicity detection kit, Roche). The quantity of LDH was measured was evaluated in a microplate reader Dygiscan (Asys Hitech GmbH), at 492 and 620 nm. Controls were taken as 100% viability. All Compounds 1 to 22 were tested for toxicity at a concentrations of 10⁻⁵ and 10⁻⁶ M, and resulted as non-toxic.

1. A compound of formula (I) 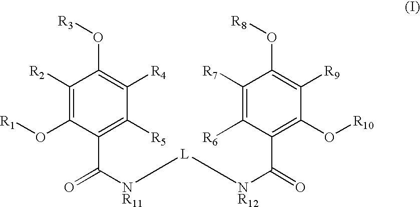
wherein
R₁ and R₁₀ are independently selected from hydrogen, substituted or unsubstituted alkyl, substituted or unsubstituted alkenyl, —(CH₂)ₘ—(CO)—Rₐ, —(CH₂)ₘ—(CO)—O—Rₐ or —(CH₂)ₘ—O—Ra, wherein m is an integer selected from 1 or 2 and Rₐ is selected from hydrogen, substituted or unsubstituted alkyl, substituted or unsubstituted cycloalkyl, substituted or unsubstituted alkenyl, substituted or unsubstituted aryl, substituted or unsubstituted aralkyl or substituted or unsubstituted heterocyclyl;
R₃ and R₈ are independently selected from hydrogen, substituted or unsubstituted alkyl, substituted or unsubstituted cycloalkyl, substituted or unsubstituted alkenyl, substituted or unsubstituted aryl, substituted or unsubstituted aralkyl, substituted or unsubstituted heterocyclyl, substituted or unsubstituted alkoxy, substituted or unsubstituted aryloxy or halogen;
R₁₁ and R₁₂ are independently selected from hydrogen, substituted or unsubstituted alkyl, substituted or unsubstituted cycloalkyl, substituted or unsubstituted alkenyl, substituted or unsubstituted aryl, substituted or unsubstituted aralkyl, substituted or unsubstituted heterocyclyl, substituted or unsubstituted alkoxy, substituted or unsubstituted aryloxy or halogen;
R₅ and R₆ are independently selected from hydrogen, C₁-C₆ alkoxy, C₁-C₆ alkyl or halogen;
R₂ and R₉ are independently selected from hydrogen, C₁-C₆ alkoxy, C₁-C₆ alkyl or halogen;
R₄ and R₇ are independently selected from hydrogen, C₁-C₆ alkoxy, C₁-C₆ alkyl or halogen;
L is a linker, consisting of a linear sequence of 1-20 units selected from —(CH₂)ₙ—, —CO—, —O—, —S—, substituted or unsubstituted arylene, cycloalkylene, heterocyclylene, or —NH—;
n=1-10;
with the provisos that:
in L, two —NH— units may not be adjacent; and
when L consists of a —(CH₂)ₙ— group then, n is 5-10;
or its enantiomers, diastereomers, tautomers, and pharmaceutically acceptable solvates and salts thereof.

2. A compound according to claim 1, wherein R₃ and R₈ are independently an C₁-C₆ alkyl.

3. A compound according to claim 2, wherein R₃ and R₈ are both methyl.

4. A compound according to claim 1, wherein the linker L consists of a —(CH₂)₅₋₁₀— group.

5. A compound according to claim 1, wherein linker L comprises a —O— unit subsequent to a substituted or unsubstituted arylene unit.

6. A compound according to claim 5, wherein the arylene unit is a substituted or unsubstituted benzylene unit.

7. A compound according to claim 6, wherein the linker L has the formula (II) 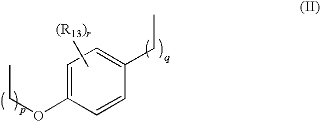
wherein R₁₃ is hydrogen or halogen, r is an integer selected from 1, 2 and 3; and p and q are integers independently selected from 1, 2, 3, 4 and 5.

8. A compound according to claim 1, wherein R₅ and R₆ are both hydrogen.

9. A compound according to claim 1, wherein R₁₁ and R₁₂ are both hydrogen.

10. A compound according to claim 1, wherein at least one of R₂, R₄, R₇ and R₉ is a halogen.

11. A compound according to claim 1, wherein R₁ is equal to R₁₀, R₂ is equal to R₉, R₃ is equal to R₈, R₄ is equal to R₇, and R₅ is equal to R₆.

12. A compound according to claim 11, wherein the linker L is symmetric, the compound having a symmetry plane.

13. A compound according to claim 1, wherein R₄ is C₁-C₆ alkoxy.

14. A compound as defined in claim 1 selected from the group consisting of: 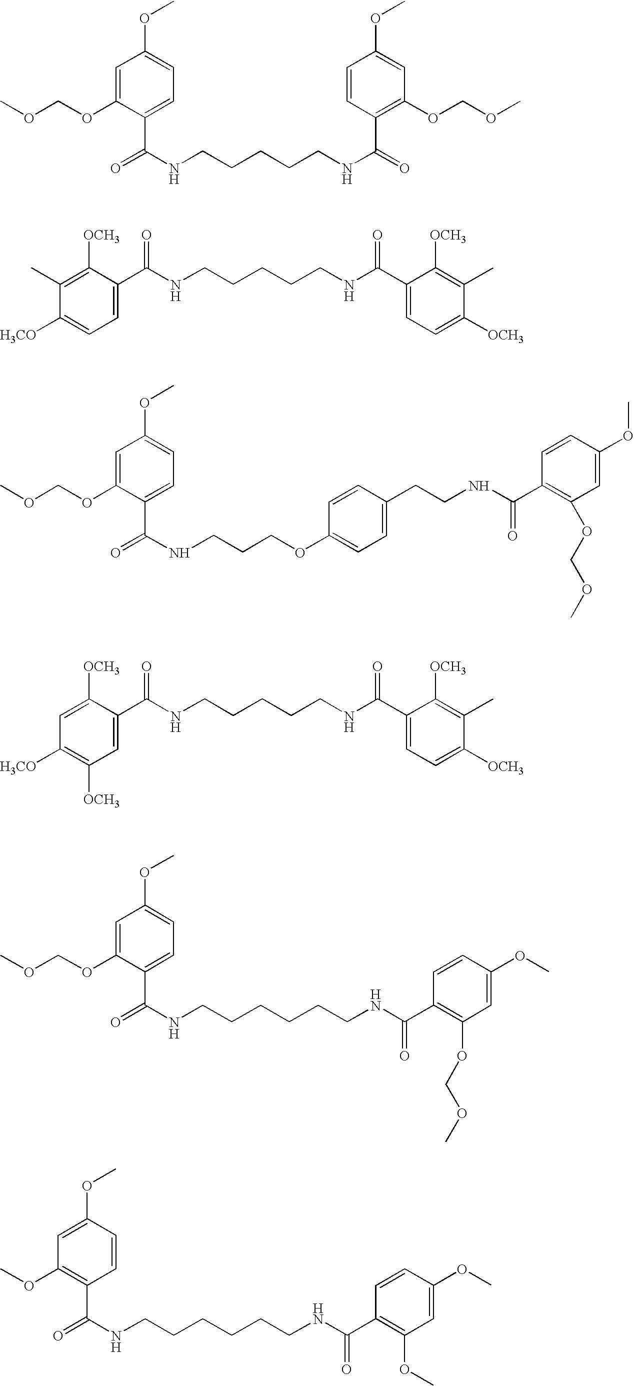 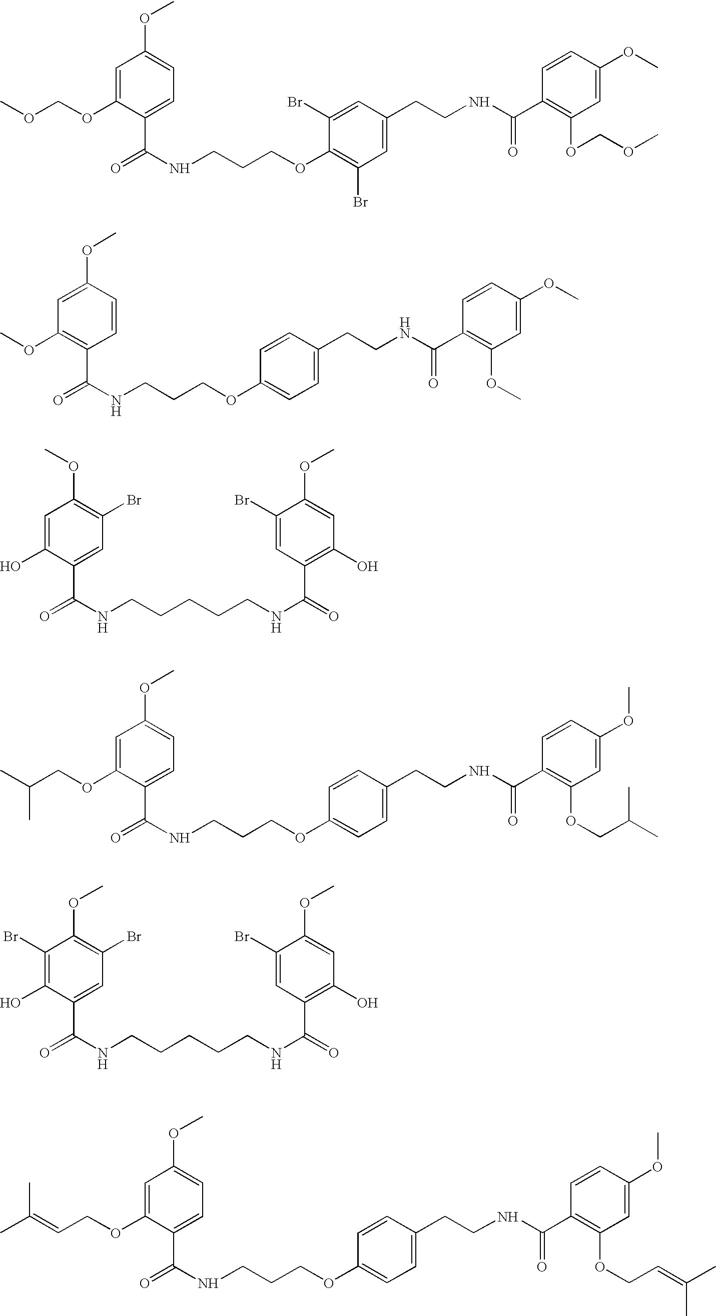 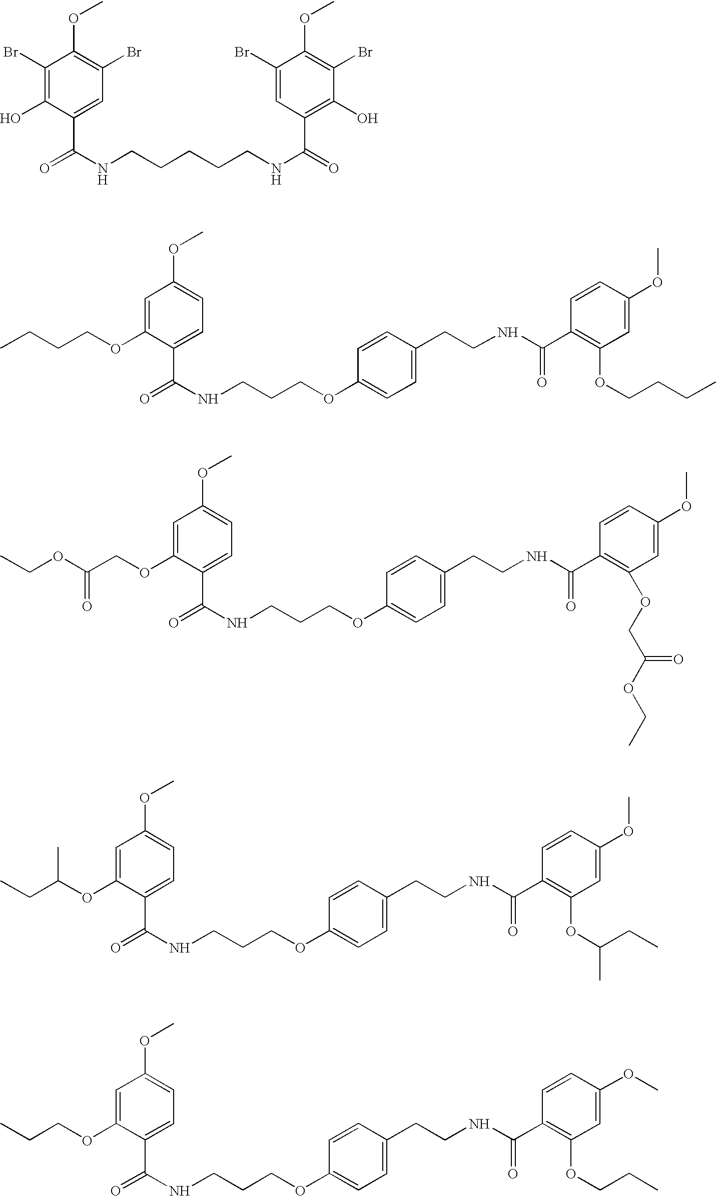 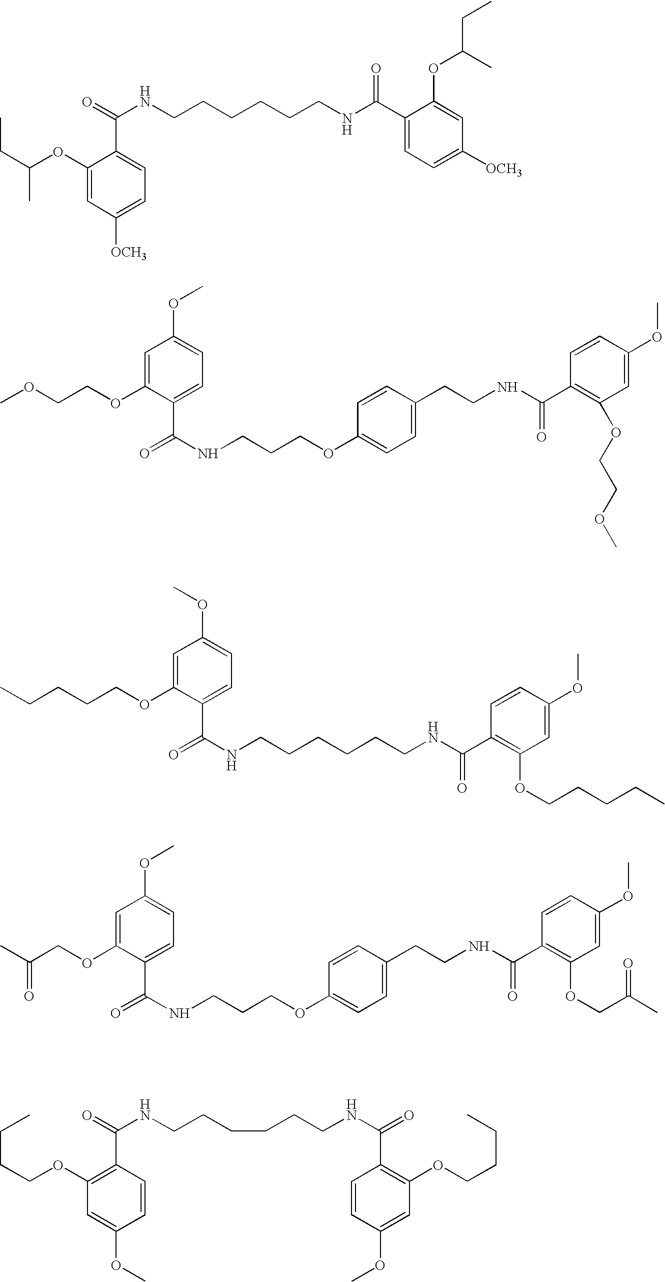
and enantiomers, diastereomers, tautomers, and pharmaceutically acceptable solvates and salts thereof.

15. A medicament comprising the compound of formula (I) as defined in claim 1.

16. A process of preparing a medicament for the treatment of a cognitive or neurodegenerative disease, said process comprising combining a compound of formula (I) of claim 1, or a pharmaceutically acceptable salt or solvate thereof, with a pharmaceutically acceptable carrier, adjuvant or vehicle.

17. The process according to claim 16, wherein the cognitive or neurodegenerative disease is selected from the group consisting of stroke, ischemia, anxiety, epilepsy, head trauma, migraine, chronic pain, neuropathic pain and acute pain, schizophrenia, depression, psychoses, drug and alcohol addiction, and neurodegenerative disorders such as Parkinson's Disease, Alzheimer's Disease, multiple sclerosis, neuropathies, Huntington's Disease and amyotrophic lateral sclerosis (ALS).

18. The process according to claim 17, wherein the neurodegenerative disease is Alzheimer's Disease.

19. The process according to claim 17, wherein the disease or condition is epilepsy.

20. A pharmaceutical composition which comprises at least one compound of formula (I) as defined in claim 1 or a pharmaceutically acceptable salt, or solvate thereof, and a pharmaceutically acceptable carrier, adjuvant or vehicle.

21. A process of using compounds as reactives for biological assays wherein said compounds comprise a compound of formula (I) as defined in claim 1.

22. Method of treating or preventing a disease or condition involving alterations of Ca²⁺ homeostasis, which method comprises administering to a patient in need of such a treatment a therapeutically effective amount of at least one compound of formula (I) as defined in claim 1 or a pharmaceutical composition thereof.
